(19) United States
(12) Patent Application Publication
(10) Pub. No.: US 2026/0078172 A1
(43) Pub. Date: Mar. 19, 2026

(54) HCV ANTIGENS AND ANTIBODIES

(71) Applicant: The Johns Hopkins University, Baltimore, MD (US)

(72) Inventors: Justin R. Bailey, Timonium, MD (US); Nicole Frumento, Baltimore, MD (US); Andrea Lynn Cox, Lutherville, MD (US)

(21) Appl. No.: 18/998,411

(22) PCT Filed: Jul. 25, 2023
(86) PCT No.: PCT/US23/28621

§ 371 Date: Jan. 24, 2025

Related U.S. Application Data
(60) Provisional application No. 63/391,986, filed on Jul. 25, 2022.

(51) Int. Cl. C07K 16/10; A61K 39/00; A61K 39/12; C07K 14/005; C12N 7/00; C12Q 1/6869; C12Q 1/70; G01N 33/576; G01N 33/68

(52) U.S. Cl. CPC C07K 16/118; A61K 39/12; C07K 14/005; C12N 7/00; C12Q 1/6869; C12Q 1/706; G01N 33/5767; G01N 33/6854; A61K 2039/575; C07K 2317/76; C12N 2770/24222; C12N 2770/24234; G01N 2333/186

(57) ABSTRACT

Methods of identifying virus antigens are provided. In particular the antigens induce broadly neutralizing antibodies in Hepatitis C virus infections. Compositions for inducing an immune response are identified by the methods described herein.

The present application claims the benefit of U.S. provisional application 63/391,986 filed Jul. 25, 2022, which is incorporated herein by reference in its entirety.

### STATEMENT REGARDING FEDERALLY SPONSORED RESEARCH

This disclosure was made with government support under grants AI127469 and AI159822 awarded by the National Institutes of Health. The government has certain rights in the invention.

### BACKGROUND

An estimated 71 million people are infected with hepatitis C virus (HCV) worldwide [1]. Given the largely asymptomatic nature of this disease, only a fraction of the HCV-infected population is aware that they are infected [2]. Of the individuals who are diagnosed, only a small percentage are effectively treated with direct-acting antiviral (DAA) therapy. Although treatment is effective and safe, it remains unavailable to many, especially in resource-limited settings, and curative treatment does not prevent reinfection [3]. Persons with chronic HCV infection are at risk for complications such as cirrhosis, end-stage liver disease, and hepatocellular carcinoma, and cirrhosis-related risks persist even after HCV cure [4, 5]. Therefore, an HCV vaccine is needed to achieve disease elimination and eradication [6, 7].

### SUMMARY

In general embodiments, methods for defining antigenic stimuli that drive the development of potent anti-HCV bNAbs in humans are provided. These methods can be utilized in identifying antigens in virus strains for the development of vaccines.

In one aspect, a method of identifying antigenic profiles of hepatitis viruses comprises measuring plasma antibody neutralization of a heterologous hepatitis virus panel; deconvoluting the plasma neutralizing antibodies to identify neutralizing antibodies having increased plasma neutralizing breadth and potency; defining an antigenic profile of infecting hepatitis virus strains associated with increased breadth and potency in hepatitis virus infected or reinfected subjects; thereby, identifying antigenic profiles of hepatitis viruses. In certain embodiments, the neutralizing breadth and potency of the identified neutralizing antibodies is compared to control reference antibodies. In certain embodiments, the neutralizing breadth and potency of the identified neutralizing antibodies is determined by neutralization assays. In certain embodiments, the neutralization assays comprise generating pseudoviral particles and incubating the pseudoviral particles with target cells to determine infectivity of the pseudoviral particles. In certain embodiments, the pseudoviral particles identified as infectious are incubated with the biological sample and determining percent neutralization values of neutralizing antibodies from each biological sample. In certain embodiments, the percent neutralization values of the neutralizing antibodies are converted to neutralization profiles of the neutralizing antibodies from each biological sample. In certain embodiments, the neutralization profiles of the neutralizing antibodies are compared to the neutralization profiles of control reference antibodies. In certain embodiments, the neutralizing antibodies are quantitated, and an antigenic profile is produced. In certain embodiments, the neutralizing antibodies are quantitated by an immunoassay. In certain embodiments, the hepatitis virus is hepatitis C virus (HCV). In certain embodiments, the immunoassay comprises antigens from hepatitis C virus strains. In certain embodiments, the biological sample is plasma.

In another aspect, a method of identifying immunogenic virus antigens comprise extracting RNA from a biological sample comprising a viral infection; quantifying and sequencing the RNA for defining specific viral strains; conducting neutralization assays by incubating pseudoviral particles with target cells to determine infectivity and incubating the pseudoviral particles with a biological sample for determining percent neutralization for each biological sample; converting of percent neutralization values to a neutralization profile for each biological sample; averaging of plasma neutralization profiles to generate a final neutralization profile for each biological sample and comparing the neutralization profiles to reference profiles; conducting a deconvolution analysis and selecting clones from various viremic time points; quantitating antibody binding to viral antigens; thereby, identifying immunogenic virus antigens. In certain embodiments, the immunogenic virus antigens are utilized in producing a vaccine. In certain embodiments, the virus is a hepatitis virus. In certain embodiments, the virus is a hepatitis C virus (HCV). In certain embodiments, the biological sample comprising an HCV infection is selected from earliest viremic and last viremic timepoints for each infection. In certain embodiments, the method further comprises identifying neutralizing antibodies having increased plasma neutralizing breadth and potency as compared to control reference antibodies. In certain embodiments, the neutralizing breadth and potency of the identified neutralizing antibodies is determined by neutralization assays. In certain embodiments, the neutralization assays comprise generating pseudoviral particles and incubating the pseudoviral particles with target cells to determine infectivity of the pseudoviral particles. In certain embodiments, the pseudoviral particles identified as infectious are incubated with the biological sample and determining percent neutralization values of neutralizing antibodies from each biological sample. In certain embodiments, the neutralization profiles of the neutralizing antibodies are compared to the neutralization profiles of control reference antibodies. In certain embodiments, the neutralizing antibodies are quantitated by an immunoassay. In certain embodiments, the immunoassay comprises HCV envelope antigens. In certain embodiments, a vaccine is produced comprising one or more immunogens.

In another aspect, a vaccine or immunogenic composition is provided that comprises a polypeptide comprising a hepatitis C virus (HCV) epitope which can induce an HCV neutralizing antibody, wherein the antibody is identified by a method embodied or disclosed herein.

In another aspect, a vaccine or immunogenic composition is provided that comprises a polypeptide comprising a hepatitis C virus (HCV) epitope which can induce an HCV neutralizing antibody. In certain embodiments, the polypeptide comprises one or more substitutions in the HCV clade 1 E1E2 protein. In certain embodiments, the one or more amino acid substitutions comprise substitutions at one or more positions 431, 482, 500 of HCV clade 1 E1E2 polypeptide. In certain embodiments, the one or more amino acid substitutions comprise substitutions at each of positions 431, 482, 500 of HCV clade 1 E1E2 polypeptide. In certain embodiments, the one or more substitutions in the HCV clade 1 E1E2 polypeptide comprises E431D, E482Q or the combination thereof.

In another aspect, a polypeptide is provided that comprises a hepatitis C virus (HCV) epitope which can induce an HCV neutralizing antibody, and wherein the polypeptide comprises one or more amino acid substitutions at one or more positions 431, 482, 500 of HCV clade 1 E1E2 polypeptide. In certain embodiments, the one or more amino acid substitutions comprise substitutions at each of positions 431, 482, 500 of HCV clade 1 E1E2 polypeptide. In certain embodiments, the one or more substitutions in the HCV clade 1 E1E2 polypeptide comprises E431D, E482Q or the combination thereof.

In another aspect, a hybrid cell is provided for, for producing an HCV neutralizing antibody.

In another aspect, a method of treating a subject infected with a hepatitis C virus (HCV) is provided that comprises administering to the subject a therapeutically effective amount of HCV neutralizing antibodies identified by the methods embodied herein.

In another aspect, a method of treating a subject infected with a hepatitis C virus (HCV) is provided that comprises administering to the subject a therapeutically effective amount of an immunogenic composition or vaccine as disclosed herein.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “about” in the context of a numerical value or range means±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, an “antibody” may be a polyclonal antisera or monoclonal antibody. The term antibody may include any of the various classes or sub classes of immunoglobulin (e.g., IgG, IgA, IgM, IgD, or IgE derived from any animal, e.g., any of the animals conventionally used, e.g., sheep, rabbits, goats, or mice, or human), e.g., the antibody comprises a monoclonal antibody, e.g., an HCV monoclonal antibody. An “isolated antibody,” as used herein, is intended to refer to an antibody that is substantially free of other antibodies having different antigenic specificities (e.g., an isolated antibody that specifically binds HCV and is substantially free of antibodies that specifically bind antigens other than HCV). Moreover, an isolated antibody may be substantially free of other cellular material and/or chemicals. The terms “monoclonal antibody” or “monoclonal antibody composition” as used herein refer to a preparation of antibody molecules of single molecular composition. A monoclonal antibody composition displays a single binding specificity and affinity for a particular epitope.

An “antibody fragment” comprises a portion of an intact antibody, preferably the antigen binding and/or the variable region of the intact antibody. Non-limiting examples of antibody fragments include Fab, Fab*, F(ab′)₂ and Fv fragments; diabodies; linear antibodies; single-chain antibody molecules and multispecific antibodies formed from antibody fragments.

A “humanized” antibody is an antibody in which all or substantially all complementarity determining region (CDR) amino acid residues are derived from non-human CDRs and all or substantially all framework region (FR) amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

By the term “synthetic antibody” as used herein, is meant an antibody, which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned), synthesizing the RNA, or other technology, which is available and well known in the art.

As used herein, an “adjuvant” refers to a substance that enhances the body's immune response to an antigen or a vaccine and may be added to the formulation that includes the immunizing agent. Adjuvants provide enhanced immune response even after administration of only a single dose of the vaccine. Adjuvants may include, for example, aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or an acrylic acid polyol cross-linked polymer. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers comprise nonionic surfactants, in particular esters of sorbitan, of mannide (e.g., anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the PLURONIC™ brand products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). In a preferred embodiment the adjuvant is at a concentration of about 0.01 to about 50%, at a concentration of about 2% to 30%, at a concentration of about 5% to about 25%, at a concentration of about 7% to about 22%, and at a concentration of about 10% to about 20% by volume of the final product. Examples of suitable adjuvants are described in U.S. Patent Application Publication No. US2004/0213817 A1. “Adjuvanted” refers to a composition that incorporates or is combined with an adjuvant.

As used herein, “antigen” is defined as a molecule that provokes an immune response. This immune response may involve either antibody production, or the activation of specific immunologically competent cells, or both. For example, any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “disease” refers to any deviation from the normal health of a mammal and includes a state when disease symptoms are present, as well as conditions in which a deviation (e.g., hepatitis C virus (HCV)) has occurred, but symptoms are not yet manifested.

The term “epitope” as used herein refers to a sequence of at least about 3 to 5, at least about 5 to 10 or 15, and not more than about 1,000 amino acids (or any integer value between 3 and 1,000), which define a sequence that by itself or as part of a larger sequence, binds to an antibody generated in response to such sequence. There is no critical upper limit to the length of the fragment, which may comprise nearly the full-length of the protein sequence, or even a fusion protein comprising two or more epitopes from the HCV polyprotein. An epitope for use in the subject disclosure is not limited to a polypeptide having the exact sequence of the portion of the parent protein from which it is derived. Indeed, viral genomes are in a state of constant flux and contain several variable domains which exhibit relatively high degrees of variability between isolates. Thus, the term “epitope” encompasses sequences identical to the native sequence, as well as modifications to the native sequence, such as deletions, additions and substitutions (generally conservative in nature).

As used herein, the term “immune response,” means a process involving the activation and/or induction of an effector function in, by way of non-limiting examples, a T cell, B cell, natural killer (NK) cell, and/or an antigen-presenting cell (APC). Thus, an immune response, as would be understood by the skilled artisan, includes, but is not limited to, any detectable antigen-specific activation and/or induction of a helper T cell or cytotoxic T cell activity or response, production of antibodies, antigen presenting cell activity or infiltration, macrophage activity or infiltration, neutrophil activity or infiltration, and the like.

An “immunological response” to an HCV antigen (including both polypeptide and polynucleotides encoding polypeptides that are expressed in vivo) or composition is the development in a subject of a humoral and/or a cellular immune response to molecules present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4⁺ and CD8⁺ T-cells.

As used herein, the term “sample” refers to a biological sample suitably obtained for the purpose of evaluation in vitro. The sample suitably may comprise a body fluid. In some embodiments, the body fluid includes, but is not limited to, whole blood, plasma, serum, lymph, breast milk, saliva, mucous, semen, cellular extracts, inflammatory fluids, cerebrospinal fluid, vitreous humor, tears, vitreous, aqueous humor, or urine obtained from the subject. In some aspects, the sample is a composite panel of two or more body fluids. In exemplary aspects, the sample comprises blood or a fraction thereof (e.g., plasma, serum, or a fraction obtained via leukapheresis).

As used herein, the terms “subject,” “patient,” “individual,” etc. are not intended to be limiting and can be generally interchanged. That is, an individual described as a “patient” does not necessarily have a given disease but may be merely seeking medical advice. For example, a “patient” or “subject” or “subject in need thereof” refers to a living member of the animal kingdom suffering from or who may suffer from HCV. In certain embodiments, the subject is a human.

The terms “therapeutically effective amount,” “treatment effective amount” and “effective amount” as used herein are synonymous unless otherwise indicated, and mean an amount of a compound, peptide or composition of the present disclosure that is sufficient to improve the condition, disease, or disorder being treated and/or achieved the desired benefit or goal (e.g., control of body weight). Those skilled in the art will appreciate that the therapeutic effects need not be complete or curative, as long as some benefit is provided to the subject. Determination of a therapeutically effective amount, as well as other factors related to effective administration of a compound of the present disclosure to a subject of this disclosure, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective treatment regimen for a subject of this disclosure is within the level of ordinary skill in the medical or veterinarian arts. In clinical use, an effective amount may be the amount that is recommended by the U.S. Food and Drug Administration, or an equivalent foreign agency. The amount of active ingredient that can be combined with the carrier materials to produce a single dosage form varies depending upon the subject being treated and the particular mode of administration.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder, or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Genbank and NCBI submissions indicated by accession number cited herein are incorporated herein by reference.

Any compositions or methods provided herein can be combined with one or more of any of the other compositions and methods provided herein.

The entire disclosure of Frumento et al., J. Clin Invest, 2022 Aug. 1, 132 (15): e160058 is fully incorporated herein in its entirety.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D are graphs demonstrating viral loads of HCV infected participants with (FIG. 1A) cleared reinfection, (FIG. 1B) persistent reinfection, (FIG. 1C) persistent infection with 1 strain, and (FIG. 1D) persistent infection with a strain switch. The study was designed for monthly viral load testing, with more than 8 years of followup in some individuals. Dashed line indicates limit of detection (LOD) of the HCV RNA assay. Infections are shaded with different colors based on the HCV subtype of the infecting virus, with subtype indicated, determined by sequencing of Core-E1 genes.

FIGS. 2A and 2B are tables demonstrating the neutralizing breadth and potency of longitudinal plasma samples. FIG. 2A: Percent neutralization of 19 HCVpp by plasma of reinfection clearance, reinfection persistence and persistence strain switch subjects. FIG. 2B: Percent neutralization of 19 HCVpp by plasma of persistence 1 strain subjects. Subjects from each group are arranged from highest to lowest neutralizing breadth. Negative percent neutralization values were converted to 0. DOV=days of viremia, calculated by counting viremic periods and excluding the period of aviremia between infections. Infxn # (gt)=number of genetically distinct infections the subject has experienced (genotype of the current infection). Breadth=number of HCVpp neutralized at least 25% by plasma at 1:100 dilution. Potency=highest percent neutralization across the panel of 19 HCVpp by plasma at 1:100 dilution. Values are the average of two independent experiments performed in duplicate Panel of 19 genotype 1 HCVpp includes strains 1a09, 1a31, 1a38, 1a53, 1a72, 1a80, 1a116, 1a123, 1a129, 1a142, 1a154, 1a157, 1b09, 1b14, 1b21, 1b34, 1b38, 1b52, 1b58 (from left to right).

FIGS. 3A-3C are a series of tables and graphs demonstrating that plasma deconvolution reveals mAb-types contributing to plasma neutralizing breadth and potency. FIG. 3A: Values shown are the proportion of each plasma neutralizing response attributed to each reference mAb. Reference mAbs are divided in narrow-breadth and broadly neutralizing (top). Proportions greater than each reference mAb's threshold are shown and marked with different colors for each NAb-type, with higher values shaded darker. Plasma samples are grouped by subject outcome. P values are for the Pearson correlation between the plasma sample neutralization profile and the best fit combined reference mAb neutralization profile. Only subjects with breadth greater than or equal to 4 were deconvoluted and included in the figure. FIG. 3B: Number of mAb types detected per subject is the same across different number of infections. Median with IQR shown. FIG. 3C: The proportion of subjects that have each mAb-type (or 0 mAb-types) after 1 infection (n=17), after two infections (n=8), and after >2 infections (n=4). Reference mAbs are divided in broadly neutralizing (purple) and narrow breadth (blue).

FIGS. 4A and 4B are a series of plots and tables demonstrating the duration of infection and multiple infections are associated with increased breadth and potency. FIG. 4A: Poisson regression analysis for breadth. Curves and 95% confidence interval (shaded areas) for each number of infections are shown. The table (right) shows the estimated coefficients and 95% confidence interval. FIG. 4B: Linear regression analysis for potency. Curves and 95% confidence interval (shaded areas) for each number of infections are shown. The table (right) shows the estimated coefficients and 95% confidence interval.

FIGS. 5A and 5B are a series of plots and tables demonstrating the genetic divergence from T/F virus is not associated with increased breadth and potency. FIG. 5A: Table illustrating divergence from infection 1 T/F virus for reinfection subjects. 5 kb hemigenomic HCV nucleotide sequences were obtained by single-genome amplification from plasma at longitudinal time points throughout the course of infection. Amino acid P distance of longitudinal E1E2 sequences from the first infection T/F virus E1E2 sequence obtained on MEGA. FIG. 5B: Breadth Poisson regression (left) and potency linear regression (right) analyses with divergence. Curves and 95% confidence interval (shaded areas) for each number of infections are shown. The table (below) shows the estimated coefficients and 95% confidence interval.

FIGS. 6A and 6B are a series of graphs, tables and a heat map demonstrating that longitudinal E1E2 FIG. 6. Longitudinal E1E2 isolates from reinfection subjects cluster in antigenically distinct clades, and infection with viruses from antigenic clade 1 is associated with increased neutralizing breadth and potency. A) Heat map illustrating ELISA binding of a panel of mAbs recognizing conformational epitopes to longitudinal E1E2 proteins from reinfection subjects. Each value is the average of 2 replicates and is normalized for binding of HCV-1, a control mAb recognizing a linear epitope that is 100% conserved across all isolates. Grey cells indicate missing data. E1E2 proteins were clustered based on mean squared distance between binding profiles. *=mAb-types or recombinant unmutated ancestors (rua) of mAb-types identified in broadly neutralizing plasma after multiple infections (FIGS. 3A-3C). FIGS. 6B and 6C: Poisson regression (FIG. 6B) and linear regression (FIG. 6C) analyses for the association of number of infections with viruses from antigenic clade 1, total number of infections, and days of viremia with neutralizing breadth (FIG. 6B) or neutralizing potency (FIG. 6C). Each total number of infections from 1-4 is illustrated on a separate graph. Curves and 95% predictive intervals (PI, shaded areas) for each number of antigenic clade 1 infections are shown in different colors. Model equations and tables with the estimated coefficients and 95% predictive intervals for each variable are shown.

FIG. 7 is a plot demonstrating the correlation between breadth of plasma (n=57) measured with the panel of 19 genotype 1 HCVpp and breadth of the same plasma measured with an antigenically diverse panel of 17 HCVpp which includes multiple genotypes. Breadth=number of HCVpp neutralized at least 25% by plasma at 1:100 dilution. Breadth for each plasma sample was normalized to account for the number of HCVpp present in each panel.

FIGS. 8A and 8B are a series of graphs showing the comparison of plasma neutralizing breadth and potency between subjects who clear and subjects who persist. FIG. 8A: Breadth (left) and potency (right) of plasma samples from subjects who clear at the time of clearance of primary infection compared to days of viremia time-matched plasma samples from subjects who persist. FIG. 8B: Breadth (left) and potency (right) of plasma samples from subjects who clear at the time of clearance of their last infection compared to days of viremia time-matched plasma samples from subjects who persist. Breadth data is not normally distributed, while potency data is normally distributed (Shapiro-Wilk test) Unpaired t test with Welch's correction was run for potency (p-value>0.05) and Mann Whitney nonparametric test was run for breadth (p-value>0.05).

FIG. 9 is a table demonstrating the true positive deconvolution threshold value for each reference mAb with single mAb and mAb combination spike-in experiments. The reference neutralization profile for each mAb was determined by measuring neutralization of the panel of 19 HCVpp in five independent experiments and averaging the neutralization profiles (rank order of sensitivity of 19 HCVpp) across all experiments. Single mAb spike-in ‘test’ data for input into the deconvolution algorithm was generated by averaging the neutralization profiles from every combination of two of the five independent experiments for each mAb in a pairwise manner (10 ‘test’ neutralization profiles for each mAb). These 10 ‘test’ neutralization profiles were then used as input into the deconvolution algorithm. mAb combination ‘test’ neutralization profiles for input into the deconvolution algorithm were generated by averaging the neutralization profiles from two independent neutralization experiments with 2 or 3 mAbs mixed at equal concentrations. Values in gray are the highest deconvolution values for each spike-in experiment. The true positive threshold for each mAb was determined by calculating the mean+2 standard deviations of all false positive values for each reference mAb across all experiments.

FIG. 10 is a table demonstrating the consistency of HEPC74 neutralization results across independent experiments was determined by performing deconvolution analysis on each independent experiment. HEPC74 was included in each plasma neutralizing breadth experiment as positive control. Values are the proportion of the neutralization activity of each sample attributed to each reference mAb by the deconvolution analysis. Data from a neutralization experiment was discarded unless HEPC74 displayed neutralizing breadth of 18 (+/−1 standard deviation) and the HEPC74 deconvolution demonstrated no false positive results and exceeded the true positive cutoff for HEPC74 (0.23).

FIG. 11 is a table demonstrating the concordance between plasma deconvolution and mAbs isolated from B cells of the same subject. Values are proportion of the neutralizing activity of each plasma sample attributed to each reference mAb by the deconvolution analysis. Values that exceed the deconvolution true positive threshold are underlined. We isolated mAbs from two study subjects (C110 and C18) which are concordant with the deconvolution results for these subjects. HEPC74 was isolated from C110's B cells. HEPC108, HEPC146, and HEPC112 were isolated from C18's B cells.

FIGS. 12A, 12B are plots demonstrating model checking. FIG. 12A: Breadth model checking for Poisson (left) and linear (right) regression model was computed in R. Residuals are symmetric and scattered for both models, but they have smaller variance in the Poisson regression model. Therefore, the Poisson model fits the data better. Equations are shown (below). FIG. 12B: Potency model checking for linear regression model. The residuals scattered and well spread. There are no obvious trends for concentration of the residuals. Therefore, the linear model fits the data well. Equation is shown (below).

FIG. 13 is a series of graphs demonstrating that no difference in neutralizing breadth or potency in participants reinfected with a virus from the same or different subtype relative to primary infection virus. Nonparametric tests for significance were conducted. Horizontal lines indicate medians.

FIGS. 14A-14F are a series of titration binding curves of reference mAbs with E1E2 lysates. Binding of reference mAbs detected by deconvolution analysis in the plasma of reinfection subjects C110 (A), C152 (B), C18 (C), C133 (D), C48 (E), and C112 (F) to longitudinal E1E2 lysates from the same subjects in an ELISA. Each binding measurement was obtained in duplicate and averaged. Error bars indicate standard deviations.

FIG. 15 is a titration curve EC50 of reference mAb binding to longitudinal E1E2 lysates correlate significantly with normalized OD results testing of the same mAb-E1E2 combinations at a single mAb concentration. Pearson correlation is shown. Each mAb titration binding measurement was obtained in duplicate and averaged (FIGS. 14A-14F). Each single mAb concentration binding is the average of 2 OD measurements in an ELISA and is normalized by HCV-1 binding (FIG. 6A).

FIG. 16 is a schematic demonstrating that antigenic clades contain E1E2 proteins from multiple genotypes and/or subtypes. Maximum Likelihood phylogenetic tree of nucleotide sequences of the same E1E2 isolates from reinfection subjects used in antigenic clustering analysis in FIGS. 6A-6C. E1E2s are color-coded by antigenic clade. All distances are drawn to scale. E1E2 subtype is indicated next to each group. Bootstrap values are indicated. Tree is rooted on bole1a.

FIGS. 17A and 17B are a series of graphs and a table demonstrating that the number of infections with viruses from antigenic clade 1 are significantly correlated with neutralizing breadth. FIG. 17A: Table summarizing the number of infections with viruses from antigenic clusters 1-4 and from distinct antigenic clusters for reinfection subjects, and the neutralizing breadth of plasma at the same timepoints. FIG. 17B: Number of infections with viruses from antigenic clade 1 is significantly associated with greater neutralizing breadth, but number of infections with viruses from clades 2-4 and or distinct clades are not. One-way ANOVA and nonparametric tests were conducted for normally distributed and non-normally distributed data, respectively. Horizontal lines indicate medians.

### DETAILED DESCRIPTION

Development of broadly neutralizing plasma antibodies during acute infection is associated with HCV clearance, but the viral epitopes of these plasma antibodies are unknown. Identification of these epitopes could define the specificity and function of neutralizing antibodies (NAbs) that should be induced by a vaccine.

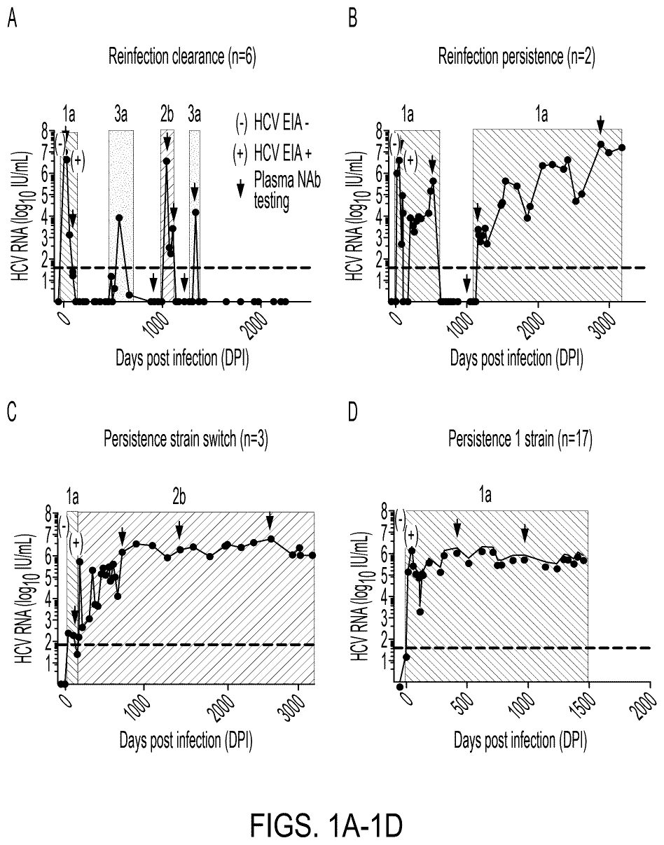

Accordingly, embodiments are directed to defining the antigenic stimuli that drive the development of potent immune responses to viruses in humans. In particular, antigenic stimuli in the generation of anti-hepatitis C virus (HCV) broadly neutralizing antibodies (bNAbs) (bNAbs) in humans were assessed. The study assessed development of bNAbs in a prospective, longitudinal cohort of persons who inject drugs (PWIDs) who acquired HCV infection during follow-up, including study participants with (1) spontaneous clearance of primary infection and multiple reinfections, (2) clearance of primary infection followed by persistent reinfection, (3) persistent, sequential infections with genetically distinct viruses, or (4) persistent, chronic infection with a single viral strain (FIGS. 1A-1D).

### Hepatitis C Virus (HCV)

A vaccine to prevent hepatitis C virus (HCV) infection is urgently needed. Unfortunately, HCV vaccine development has been hampered by incomplete understanding of correlates of protective immunity in humans and by inadequate methods for assessing antibody responses induced by candidate vaccines. Around 25% of individuals who are acutely infected with HCV spontaneously clear the infection without treatment. This spontaneous clearance is associated with early development of broadly neutralizing plasma, suggesting that identification of neutralizing antibodies (NAbs) present in the plasma of these individuals could elucidate the NAbs that should be induced by a vaccine. However, the antigenic stimuli required to induce NAbs present in broadly neutralizing plasma are unknown.

One of the major challenges to the development of a successful HCV vaccine is the extraordinary genetic diversity of the virus [8-10]. Fortunately, multiple broadly neutralizing antibodies (bNAbs) have been identified that block infection by diverse HCV strains in vitro, and infusion of bNAbs is protective against HCV infection in animal models [11-17]. In humans, along with potent antiviral T cell responses, early development of plasma bNAbs has been associated with spontaneous clearance of primary HCV infection, which occurs in about 25% of infected individuals [18-20]. Notably these immune responses do not provide sterilizing immunity since individuals can be reinfected after HCV clearance. However, about 80% of those who clear their first infection clear subsequent reinfections [19]. Reinfections are associated with a rapid rise in neutralizing antibody (NAb) titers, shorter duration of infection, and lower peak viremia, which indicate protection by adaptive immunity [21]. Thus, individuals who clear multiple reinfections can serve as a model for a desired vaccine response.

However, questions remain about how anti-HCV bNAbs are induced in humans. Central to vaccine design, it remains unclear whether multivalent and/or prime-boost immunizations are needed to induce anti-HCV bNAbs, and what genetic or antigenic criteria should be used to select vaccine antigens. There is evidence that sequential exposure to dengue, influenza, or HIV antigens can lead to broader immune responses [22-24]. For chronically HCV-infected individuals, longer duration of infection leads to greater neutralizing breadth [19, 25-27]. While reinfection after HCV clearance can lead to broadening of the neutralizing antibody response [25], not all reinfected individuals develop bNAbs, and the genetic or antigenic features of primary and reinfecting viruses associated with acquisition of bNAbs have not been defined. For example, it is unclear whether exposure to multiple highly diverse variants or reinfection with antigenically similar viruses is required to induce a potent bNAb response. Characterization of these genetic and antigenic features would directly inform development of a prophylactic HCV vaccine.

### Methods of Identifying HCV Antigens

Provided herein are methods for defining antigenic stimuli that drive the development of potent anti-HCV bNAbs in humans are provided. These methods can be utilized in identifying antigens in virus strains for the development of vaccines.

The method includes measuring plasma antibody neutralization of a heterologous hepatitis virus panel; deconvoluting the plasma neutralizing antibodies to identify neutralizing antibodies having increased plasma neutralizing breadth and potency; defining an antigenic profile of infecting hepatitis virus strains associated with increased breadth and potency in hepatitis C virus infected or reinfected subjects; thereby, identifying antigenic profiles of hepatitis C viruses.

In some embodiments, neutralization of an HCV infection is based on a HCV pseudotyped particles (HCVpp) neutralization assay. HCVpp consist of unmodified HCV envelop glycoproteins assembled onto retroviral or lentiviral core particles. HCVpp infect hepatoma cell lines and hepatocytes in an HCV envelop protein-dependent matter. The presence of a marker gene packaged within the HCVpp allows fast and reliable determination of antibody-mediated neutralization. In some embodiments, neutralization of an HCV infection is based on a recombinant cell culture-derived HCV (HCVcc) neutralization assay infecting human cell lines.

The methods of identifying epitopes that generate Hepatitis C virus (HCV) neutralizing antibodies also include a neutralization profile that comprises a ranking of relative neutralization of each HCVpp and of each biological sample. In other examples, the reference antibody neutralization profiles are added in various proportions to generate an array of possible combined antibody neutralization profiles. For example, a specific combined reference antibody neutralization profile is correlated with each plasma neutralization profile to identify the proportion of each reference antibody contributing to the neutralization profile of the biological sample.

The methods described herein further provide that the neutralization profiles identify individual antibodies which bind to distinct HCV epitopes or are cross-reactive to related HCV epitopes. Moreover, the methods also including isolating the HCV neutralizing antibodies. For example, the method may include comprises culturing the host cell and isolating from the culture an antibody binding the HCV epitope. The method can further comprise screening the antibody in a cell culture system or in vivo to determine that it is a neutralizing antibody.

The methods described herein are also suitable for scaling, including, for example as a high throughput method

### Vaccine

The methods herein also provide for a vaccine including a polypeptide having an Hepatitis C virus (HCV) epitope which induces an HCV neutralizing antibody, said antibody identified by the methods described herein.

In one embodiment, the present disclosure provides an immunogenic composition for inducing an immune response against HCV in a subject, for example, in one embodiment, a vaccine. For a composition to be useful as a vaccine, the composition must induce an immune response against the HCV antigen in a cell, tissue or mammal (e.g., a human). In some instances, the vaccine induces a protective immune response in the mammal. As used herein, an “immunogenic composition” may comprise an antigen (e.g., a peptide or polypeptide), a nucleic acid encoding an antigen, a cell expressing or presenting an antigen or cellular component, a virus expressing or presenting an antigen or cellular component, or a combination thereof. In certain embodiments, the composition comprises or encodes all or part of any peptide antigen described herein, or an immunogenically functional equivalent thereof. In other embodiments, the composition is in a mixture that comprises an additional immunostimulatory agent or nucleic acids encoding such an agent. Immunostimulatory agents include but are not limited to an additional antigen, an immunomodulator, an antigen presenting cell, lipid nanoparticle, or an adjuvant. In other embodiments, one or more of the additional agent(s) is covalently bonded to the antigen or an immunostimulatory agent, in any combination.

In the context of the present disclosure, the term “vaccine” refers to a composition that induces an immune response upon inoculation into an animal. In some embodiments, the induced immune response provides protective immunity. A vaccine of the present disclosure may vary in its composition of nucleic acid and/or cellular components. In a non-limiting example, a nucleic acid encoding an HCV antigen might also be formulated with an adjuvant. Of course, it will be understood that various compositions described herein may further comprise additional components.

For example, one or more vaccine components may be comprised in a lipid, liposome, or lipid nanoparticle. In another non-limiting example, a vaccine may comprise one or more adjuvants. A vaccine of the present disclosure, and its various components, may be prepared and/or administered by any method disclosed herein or as would be known to one of ordinary skill in the art, in light of the present disclosure.

In various embodiments, the induction of immunity by the expression of the HCV antigen can be detected by observing in vivo or in vitro the response of all or any part of the immune system in the host against the HCV antigen. For example, a method for detecting the induction of cytotoxic T lymphocytes is well known. A foreign substance that enters the living body is presented to T cells and B cells by the action of antigen presenting cells (APCs). Some T cells that respond to the antigen presented by APC in an antigen specific manner differentiate into cytotoxic T cells (also referred to as cytotoxic T lymphocytes or CTLs) due to stimulation by the antigen. These antigen-stimulated cells then proliferate. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC and detecting the induction of CTL. Furthermore, APCs have the effect of activating B cells, CD4⁺ T cells, CD8⁺ T cells, macrophages, eosinophils and NK cells.

The induction of immunity by expression of the HCV antigen can be confirmed by observing the induction of antibody production against the HCV antigen. For example, when antibodies against an antigen are induced in a laboratory animal immunized with the composition encoding the antigen, and when antigen-associated pathology is suppressed by those antibodies, the composition is determined to induce immunity. The specificity of the antibody response induced in an animal can include binding to many regions of the delivered antigen, as well as the induction of neutralization capable antibodies that that prevent infection or reduce disease severity. For example, epitopes in the E1E2 region as identified herein.

A method for evaluating the inducing action of CTL using dendritic cells (DCs) as APC is also well known in the art. DC is a representative APC having a robust CTL inducing action among APCs. In the methods of the disclosure, the epitope of a polypeptide or peptide or combinations thereof is initially expressed by the DC and then this DC is contacted with T cells. Detection of T cells having cytotoxic effects against the cells of interest after the contact with DC shows that the epitope of a polypeptide or peptide or combinations thereof has an activity of inducing the cytotoxic T cells. Furthermore, the induced immune response can also be examined by measuring IFN-γ produced and released by CTL in the presence of antigen-presenting cells that carry immobilized peptide or a combination of peptides by visualizing using anti-IFNγ antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7. The antigens confirmed to possess CTL-inducing activity by these methods are antigens having DC activation effect and subsequent CTL-inducing activity. Furthermore, CTLs that have acquired cytotoxicity due to presentation of the antigen by APC can be also used as vaccines against antigen-associated disorders.

The induction of immunity by expression of the HCV antigen can be further confirmed by observing the induction of CD4⁺ T cells. CD4⁺ T cells can also lyse target cells, but mainly supply help in the induction of other types of immune responses, including CTL and antibody generation. The type of CD4⁺ T cell help can be characterized, as Th1, Th2, Th9, Th17, T regulatory (Treg), or T follicular helper (Tfh) cells. Each subtype of CD4⁺ T cell supplies help to certain types of immune responses.

The therapeutic compositions of the disclosure may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present disclosure, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Antigens

The present disclosure provides a composition that induces an immune response in a subject. In certain embodiments, the immune response induces bNAbs. In one embodiment, the composition comprises an HCV antigen. The antigen may include, but is not limited to a polypeptide, peptide, protein, virus, or cell that induces an immune response in a subject. In one embodiment, the composition comprises a nucleic acid sequence, which encodes an HCV antigen.

In one embodiment, the antigen comprises a polypeptide or peptide associated with HCV, such that the antigen induces an immune response against the antigen, and therefore HCV. In one embodiment, the antigen comprises a fragment of a polypeptide or peptide associated with HCV, such that the antigen induces an immune response against HCV.

In some embodiments, the HCV antigen is at least one of HCV envelope E1 protein, HCV envelope E2 protein, HCV core (C) protein, or a fragment thereof. In some aspects, the core is used to allow the formation of secreted subviral particles containing E1 and E2. In some instances, these secreted particles are a better form for presentation to B cells. In one embodiment, the antigen comprises a protein comprising a signal peptide (SP) from MHC class II. Other signal peptides that may be used include, but are not limited to, signal sequences derived from IL-2, tPA, mouse and human IgG, and synthetic optimized signal sequences.

In some embodiments, the composition comprises an mRNA comprising a nucleic acid sequence encoding E1-E2 antigens as identified herein.

In some embodiments, the composition comprising a nucleic acid sequence that encodes an HCV core protein. The HCV core protein may be of any HCV isolate.

In some embodiments, the composition comprises a nucleic acid sequence encoding C-E1-E2 or peptides thereof.

The HCV antigen may be of any type or strain of HCV. For example, in one embodiment, the HCV antigen is a protein, or fragment thereof, of a HCV strain including, but not limited to, 1a, 1b, 1c, 1e, 1g, 1 h, 1l, 2a, 2b, 2c, 2d, 2e, 2i, 2j, 2k, 2m, 2q, 2r, 3a, 3b, 3g, 3 h, 3i, 3k, 4a, 4b, 4c, 4d, 4f, 4g, 4k, 41, 4m, 4n, 40, 4p, 4q, 4r, 4t, 4v, 10 4w, 5a, 6a, 6b, 6c, 6d, 6e, 6f, 6g, 6 h, 61, 6j, 6k, 61, 6m, 6n, 60, 6p, 6q, 6r, 6s, 6t, 6u, 6v, 6w, 6xa, and 7a.

In some embodiments, the HCV antigen comprises an amino acid sequence that is substantially homologous to the amino acid sequence of a HCV antigen described herein and retains the immunogenic function of the original amino acid sequence. For example, in some embodiments, the amino acid sequence of the HCV antigen has a degree of identity with respect to the original amino acid sequence of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the HCV antigen is encoded by a nucleic acid sequence of a nucleic acid molecule. In some embodiments, the nucleic acid sequence comprises DNA, RNA, cDNA, viral DNA, a variant thereof, a fragment thereof, or a combination thereof. In one embodiment, the nucleic acid sequence comprises a modified nucleic acid sequence. In some instances, the nucleic acid sequence comprises include additional sequences that encode linker or tag sequences that are linked to the antigen by a peptide bond.

### Methods of Treating

Provided herein are methods for treating a subject infected with a Hepatitis C virus (HCV). The methods include administering to the subject a therapeutically effective amount of HCV neutralizing antibodies identified by the methods described herein. In other examples, the methods of treating include administering the vaccines described herein.

In one embodiment, the composition is administered to a subject having an infection, disease, or disorder associated with HCV. In one embodiment, the composition is administered to a subject at risk for developing the infection, disease, or disorder associated with HCV. For example, the composition may be administered to a subject who is at risk for being in contact with HCV. In one embodiment, the composition is administered to a subject who lives in, traveled to, or is expected to travel to a geographic region in which HCV is prevalent. In one embodiment, the composition is administered to a subject who is in contact with or expected to be in contact with another person who lives in, traveled to, or is expected to travel to a geographic region in which HCV is prevalent. In one embodiment, the composition is administered to a subject who has knowingly been exposed to HCV through their occupation, sexual, or other contact.

Administration of the compositions of the disclosure in a method of treatment can be achieved in a number of different ways, using methods known in the art. In one embodiment, the method of the disclosure comprises systemic administration of the subject, including for example enteral or parenteral administration. In some embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the disclosure may be administered to a subject either alone, or in conjunction with another agent. The therapeutic and prophylactic methods of the disclosure thus encompass the use of pharmaceutical compositions encoding a HCV antigen, adjuvant, or a combination thereof, described herein to practice the methods of the disclosure. The pharmaceutical compositions useful for practicing the disclosure may be administered to deliver a dose of from 1 ng/kg/day and 100 mg/kg/day. In one embodiment, the disclosure envisions administration of a dose, which results in a concentration of the compound of the present disclosure from 10 nM and 10 μM in a mammal. Typically, dosages which may be administered in a method of the disclosure to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months, several years, or even less frequently, such as every 10-20 years, 15-30 years, or even less frequently, such as every 50-100 years. The frequency of the dose will be readily apparent to the skilled artisan and will depend upon any number of factors, such as, but not limited to, the type and severity of the disease being treated, the type and age of the mammal, etc.

In some embodiments, administration of an immunogenic composition or vaccine of the present disclosure may be performed by single administration or boosted by multiple administrations.

In one embodiment, the disclosure includes a method comprising administering one or more compositions encoding one or more HCV antigens described herein. In some embodiments, the method has an additive effect, wherein the overall effect of the administering the combination is approximately equal to the sum of the effects of administering each HCV antigen or adjuvant. In other embodiments, the method has a synergistic effect, wherein the overall effect of administering the combination is greater than the sum of the effects of administering each HCV antigen or adjuvant.

In certain embodiments, the vaccines or neutralizing antibodies can be administered in combination with one or more anti-viral agents. The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration.

Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In some embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents: preservatives; physiologically degradable compositions such as gelatin; aqueous vehicles and solvents; oily vehicles and solvents; suspending agents: dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, Pa.), which is incorporated herein by reference.

### EXAMPLES

### Example 1: Repeated Exposure to Heterologous but Antigenically Related Hepatitis C Viruses is Associated with Induction of Potent and Broadly Neutralizing Antibodies

In this study, the aim was to define the antigenic stimuli that drive the development of potent anti-HCV bNAbs in humans. The development of bNAbs was assessed in a prospective, longitudinal cohort of persons who inject drugs (PWIDs) who acquired HCV infection during follow-up, including study participants with (1) spontaneous clearance of primary infection and multiple reinfections, (2) clearance of primary infection followed by persistent reinfection, (3) persistent, sequential infections with genetically distinct viruses, or (4) persistent, chronic infection with a single viral strain (FIGS. 1A-1D). The neutralizing breadth and potency of plasma antibodies was measured at multiple timepoints in each study subject and the mAb-types responsible for the neutralizing activity of each plasma sample were identified. The relationship between development of bNAbs and (1) exposure to multiple genetically distinct infections, (2) duration of viremia, (3) genetic distance between primary infection and reinfection viruses, and (4) antigenic similarity between primary infection and reinfection viruses was identified. These data were used to develop a rigorous model identifying key features of stimuli capable of inducing potent bNAbs in humans.

### Materials and Methods

Human Subjects. Plasma was obtained from the BBAASH cohort [28]. Plasma samples from persistently infected subjects were time-matched with clearance subjects based on days of viremia (DOV). DOV is calculated by only counting viremic periods and excluding the period of aviremia between infections. The first viremic timepoint and the aviremic timepoint prior to reinfections were not time-matched, and therefore not included in comparisons between groups.

Cell lines. HEK293T/17 cells (sex: female) were obtained from ATCC (cat #CRL-11268) and maintained in Dulbecco's Modified Eagle Medium and supplemented with sodium pyruvate, 10% heat inactivated fetal bovine serum, and glutamine. HEP3B cells (sex: male), were obtained from the ATCC (cat #HB-8064), and maintained in Modified Eagle Medium, supplemented with sodium pyruvate, 10% heat inactivated fetal bovine serum, nonessential amino acids, penicillin-streptomycin, and glutamine. Cells were cultured at 37 C in a humidified incubator with 5% CO₂, and monolayers were disrupted at 80% to 100% confluence with Trypsin-EDTA.

Source of Reference bNAbs. HEPC74, HEPC98, HEPC112, HEPC146, HEPC108, HEPC74RUA and HEPC108RUA were isolated by JRB, AIF and JEC [29]. MAbs CBH-2, CBH-7, HC-1 [15], HC84.26 [30], HC33.4 (Dr. Steven Foung, (Stanford University School of Medicine, Palo Alto, California). MAbs ARIA, AR3A [11], and AR4A (Dr. Mansun Law, Scripps Research Institute, La Jolla, California).

HCV Viral Load and Serology Testing. HCV viral loads (IU/mL) were quantified after RNA extraction with the use of commercial real-time reagents (Abbot HCV Real-time Assay) migrated onto a research-based real-time PCR platform (Roche 480 LightCycler). HCV seropositivity was determined using the Ortho HCV version 3.0 ELISA Test System (Ortho Clinical Diagnostics).

Viral sequencing. E1 sequences (H77 nt 943-1288) Genetic distance >0.03 used were used to define specific viral variants and was calculated with HIV-TRACE [33]. HCV hemigenomes from plasma virus were amplified by RT-PCR after limiting dilution to ensure single-genome amplification, using previously described methods (Li et al., 2012). E1E2 was PCR amplified from single-genome amplification amplicons of interest and cloned as previously described [19]. Sequences of all E1E2 clones were confirmed after cloning.

HCVpp Production, Infectivity, and Neutralization Assays. HCVpp were produced by lipofectamine-mediated transfection of HCV E1E2, pNL4-3.Luc.R-E-, and pAdVantage (Promega) plasmids into HEK293T cells as previously described [34]. For infectivity testing, HCVpp were incubated on Hep3B target cells for 5 hours before media was removed. The panel of 19 heterologous genotype 1 HCVpp has been described previously [19, 35]. All HCVpp used in neutralization assays produced RLU values at least 10-fold above background entry by mock pseudoparticles. Only HCVpp preparations producing at least 1E6 RLU were used for neutralization experiments, and HCVpp input was normalized to a maximum of 10E6 RLU. Each plasma sample was tested in duplicate to control for plate-to-plate variability and in two independent experiments using different stocks of panel of 19 HCVpp to control for batch-to-batch variability.

Neutralization assays were performed as described previously [36]. HCVpp were incubated for 1 hour with heat-inactivated plasma at a 1:50 dilution or mAb at 10 μg/mL and then added in duplicate to Hep3B target cells for 5 hours before medium was changed. Nonspecific human IgG (Sigma-Aldrich) at 100 μg/mL and heat-inactivate pre-immune plasma (PIP) at 1:50 were used as a negative control. Infection was determined after 72 hours by measurement of luciferase activity of cell lysates in RLU. Percent neutralization was calculated by the formula 1−(RLUautologousplasma/RLUautologousPIP)*100. Based on 65 independent tests performed in duplicate with a variety of different HCVpp [37], nonspecific neutralization by negative control mAb R04 at 100 mcg/mL was an average of 1.0%, with a standard deviation of 19.9%. Therefore, the cutoff for true-positive neutralization was set at >25%. Percent neutralization values were converted to a neutralization profile for each plasma sample (rank order of HCVpp neutralization with the most sensitive HCVpp ranked 1 and the least sensitive HCVpp ranked 19) for input into the deconvolution algorithm.

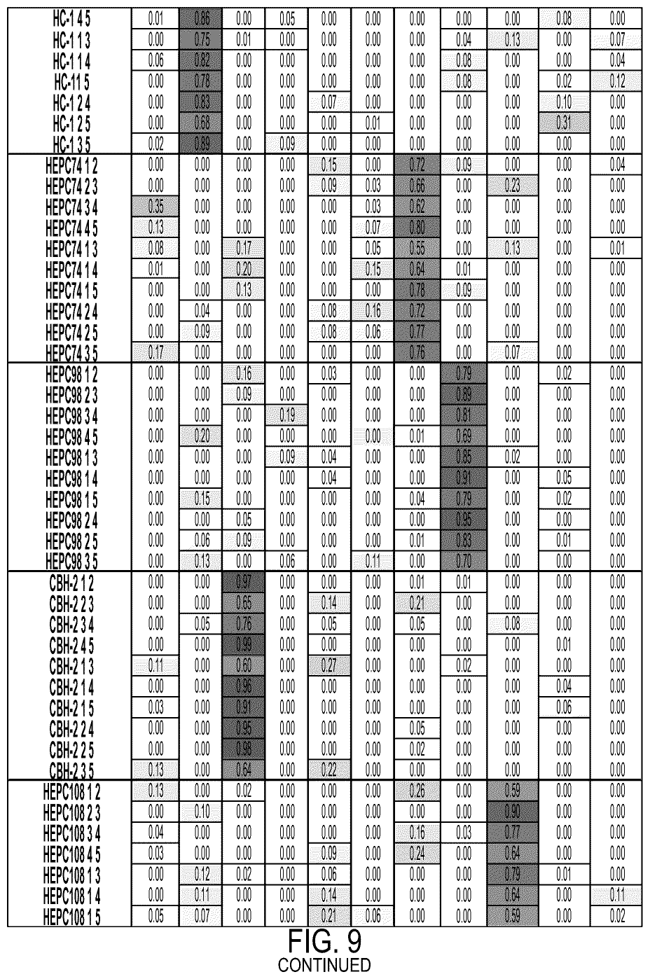

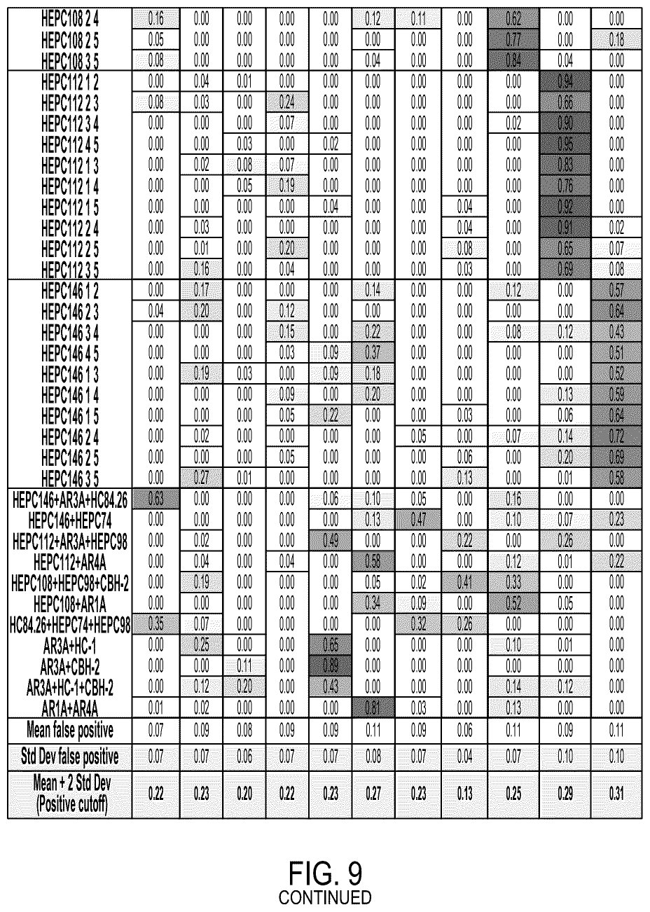

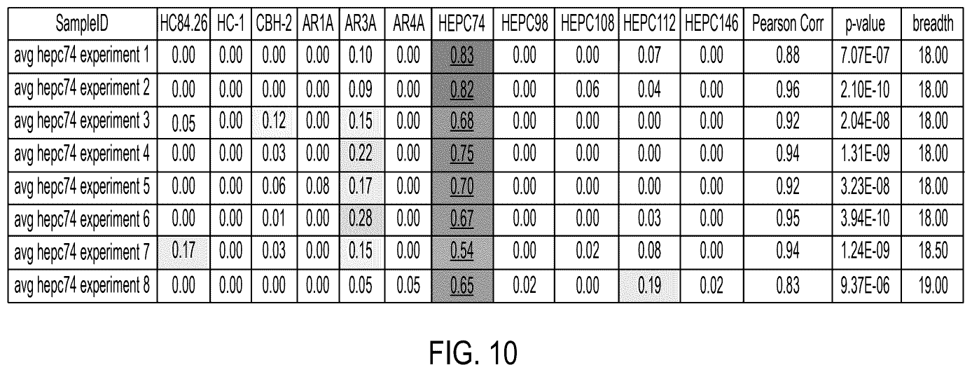

Deconvolution of mAb-types in plasma. Plasma neutralization profiles (rank order of sensitivity of 19 HCVpp to each sample) were averaged across the two independent experiments to generate a final neutralization profile for each plasma sample. Neutralization profiles of each plasma were compared to neutralization profiles of a panel of 11 reference HCV-specific mAbs. Neutralization profiles for each reference mAb were averaged across five independent experiments. Neutralization profiles for 8 reference mAbs were generated in a previous study [38]. Deconvolution analysis was performed using Pearson correlations between plasma and reference mAb neutralization profiles to delineate the relative proportion of each reference mAb present in each plasma sample, as previously described (cite Kinchen), using code written in Python. HEPC74 served as a positive control in each plasma neutralization experiment. Only plasma neutralization results were included from experiments where HEPC74 had a breadth of 18 (+/−1 standard deviation) and where deconvolution of the neutralization profile of HEPC74 obtained in that experiment resulted in a HEPC74-positive result (i.e., proportion of response attributed to HEPC74 surpassed the true positive cutoff (FIG. 10) Upload code on git.hub.

E1E2 clone selection. Clones from earliest viremic and last viremic timepoints for each infection were preferentially selected over clones from intermediate timepoints. In infections with multiple viremic timepoints, when possible, clones from the same phylogenetic lineage spanning multiple timepoints were selected. When clones from the same lineage could not be selected, a dominant clone from a dominant clade clone was selected from the dominant clade (clade with most clones) from each timepoint. Within a clade, the most abundant clone was selected, or if there was no most abundant clone, the clone closest to the MRCA for that clade was selected. For infections that were sequenced at a single timepoint, the most abundant clone was selected.

HCV E1E2 ELISA. mAb binding to E1E2 was quantitated using an ELISA as previously described [29]. Briefly, 293T cells were transfected with E1E2 expression constructs. Cell lysates were harvested at 48 hours. Plates were coated with 500 ng Galanthus nivalis lectin (EY Labs) and blocked with PBS containing 0.5% Tween-20, 1% nonfat dry milk, and 1% goat serum, and E1E2-containing cell lysates were added. For titration binding curves, MAbs were assayed in duplicate at 3-fold serial dilutions, starting at 10 μg/mL, and binding was detected with HRP-conjugated anti-human IgG secondary antibody (Vector Laboratories PI-3000). Equivalent protein concentrations in each E1E2 lysate preparation were confirmed using serial dilutions of control mAb, HCV-1. E1E2 lysate from the same subjects were assayed in the same experiment against all mAbs picked up in the deconvolution of plasma from that subject. For antigenic profiling, RUA mAbs were assayed in duplicate at 10 μg/ml, while the rest of the mAbs were assayed at 0.08 μg/ml. HCV-1 and Nonspecific human IgG (Sigma-Aldrich) were included in the antigenic profiling experiment as positive and negative control, respectively. All lysates were tested against all mAbs in the panel in the same experiment. Each mAb was tested in a separate plate against all E1E2 lysates.

Antigenic profiling data cleanup and clustering. To account for plate-to-plate variability, the absolute difference was calculated between the OD replicates for IgG binding and set a cutoff for absolute difference as average+2 standard deviation. Any OD pairs with absolute difference higher than cutoff across all mAbs were discarded. This led to 4 OD pairs being deleted for AR3A binding. To remove from the analysis any E1E2 lysates that did not express well, the true positive cutoff for OD value (average+2 standard deviation) for IgG binding was calculated and then any E1E2 lysates with average HCV-1 binding below true positive cutoff were deleted from the entire analysis. This led to 6 E1E2 lysates to be excluded from the analysis. HEPC146rua and HEPC98rua did not have detectable binding to any E1E2 proteins, so they were excluded from subsequent analyses. To account for difference in E1E2 lysate concentration, average OD values were normalized for each mAb to HCV-1 average OD for the same lysate. The resulting average OD values were entered into a code in R that clustered them based on mean squared distance between binding profiles [39].

Statistical analysis. Statistical analyses were performed in Prism (GraphPad software, v7.02). Correlations between plasma and reference mAb neutralization profiles were calculated using Pearson's method. For all comparisons, p values less than 0.05 were considered significant. Breadth and potency models were computed in R. Upload code on git.hub.

Accession number(s). The GenBank accession numbers of E1E2 clones expressed are: OK553726-OK554430, OK583165-OK583829, MZ834892-MZ835192, OK582746-OK583164, OL332220-OL332312, MZ556841-MZ556946, OK502877-OK503334, MZ457964-MZ458098, OK503618-OK504315, OK582292-OK582745.

### Results

Selection of Study Participants. Study participants were PWIDs enrolled in the Baltimore Before and After Acute Study of Hepatitis (BBAASH) cohort who were identified before or very early during acute HCV infection (prior to HCV antibody seroconversion) and subsequently followed in a study designed for monthly follow up through spontaneous clearance of primary HCV infections and reinfections or over years of chronic infection. Study participants were divided into four groups based on different patterns of infection (FIGS. 1A-1D). Reinfection clearance subjects (n=6) were defined as individuals who were infected with and subsequently cleared without treatment multiple infections with genetically distinct HCV strains (divergence between strains ≥0.03), with an interval of at least 60 days of aviremia between infections (FIG. 1A). Reinfection persistence subjects (n=2) were defined as HCV infected persons who cleared their first infection without treatment and, after an interval of at least 60 days of aviremia, were subsequently reinfected with another genetically distinct virus which was not cleared (FIG. 1B). Persistence strain switch subjects were infected sequentially with more than one genetically distinct viral strain without a detected interval of aviremia between the first and second infections (FIG. 1C). For these subjects, the first infection prior to the viral strain switch was considered the primary infection and subsequent infection after the viral strain switch as a reinfection. Finally, as controls for this study, participants who remained HCV infected with a single HCV strain (divergence between longitudinal viruses <0.03) over many years of follow-up (designated “persistence 1 strain” subjects, n=17, FIG. 1D) were selected. Overall, the subject age at the time of seroconversion, sex, race, and HCV infection genotype were not significantly different between the groups (Table 1).

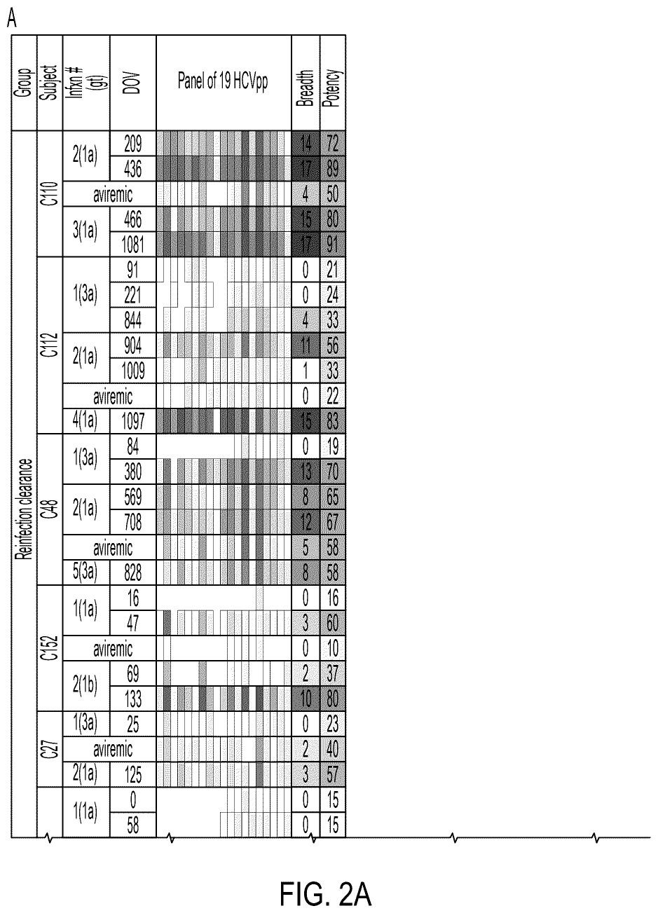

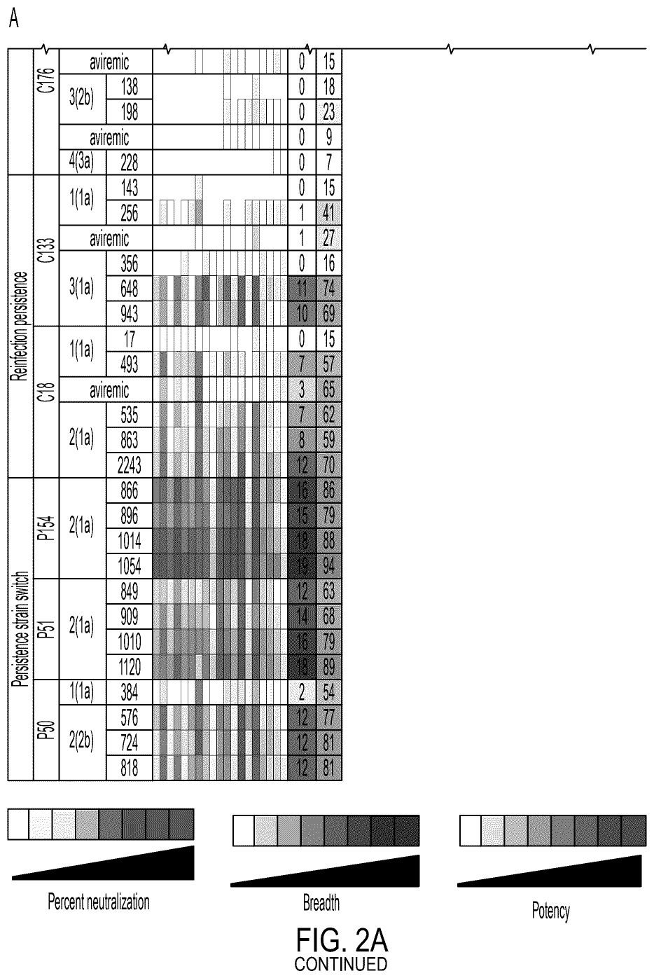

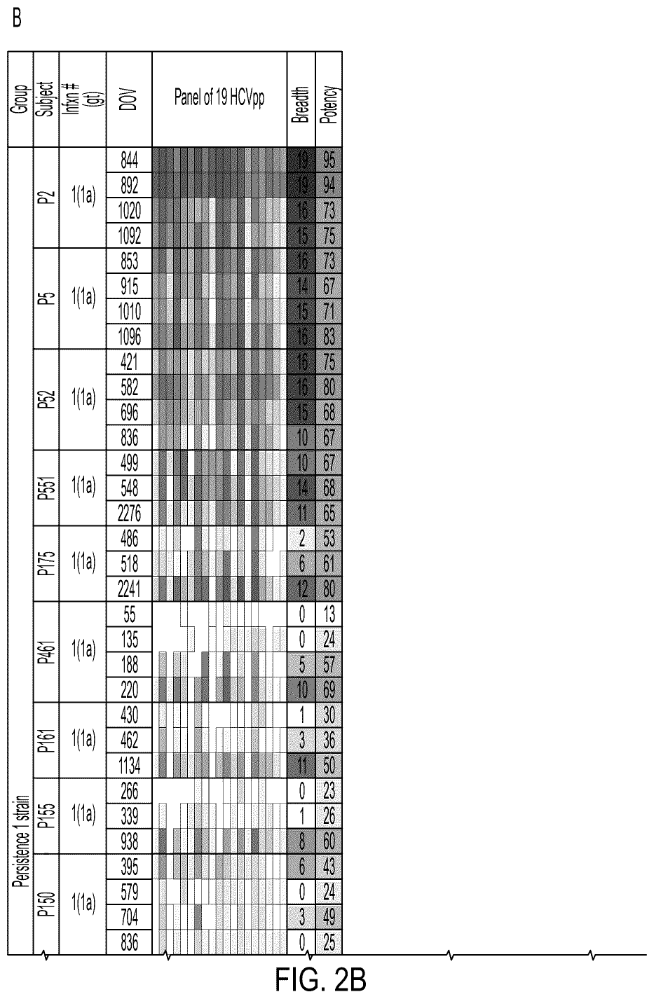

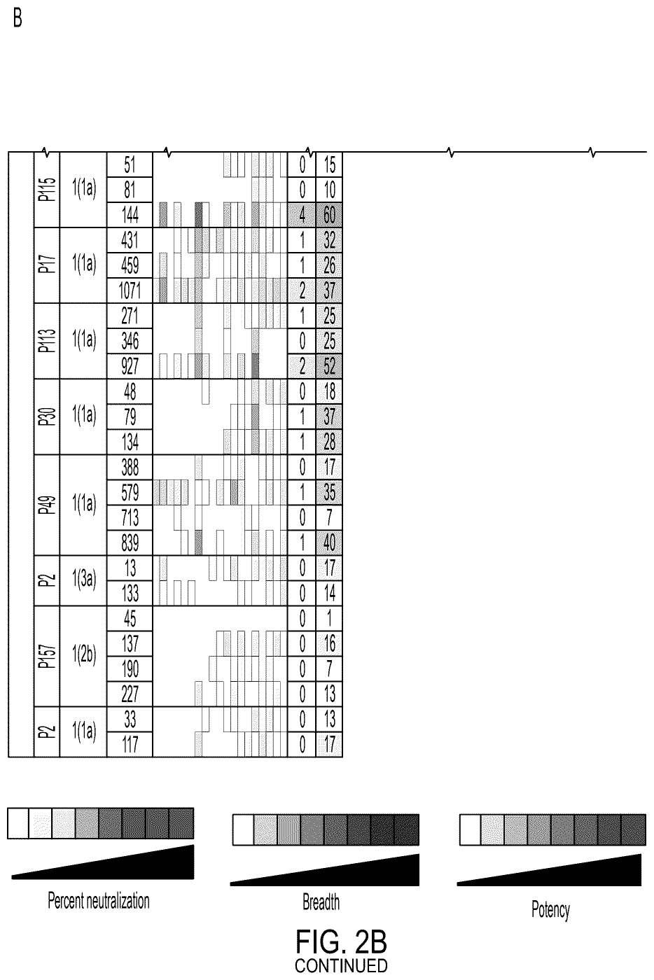

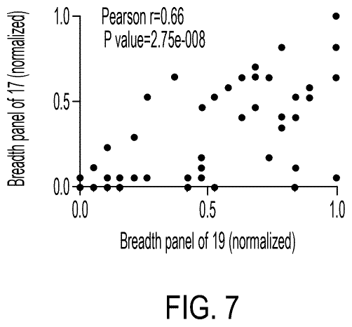

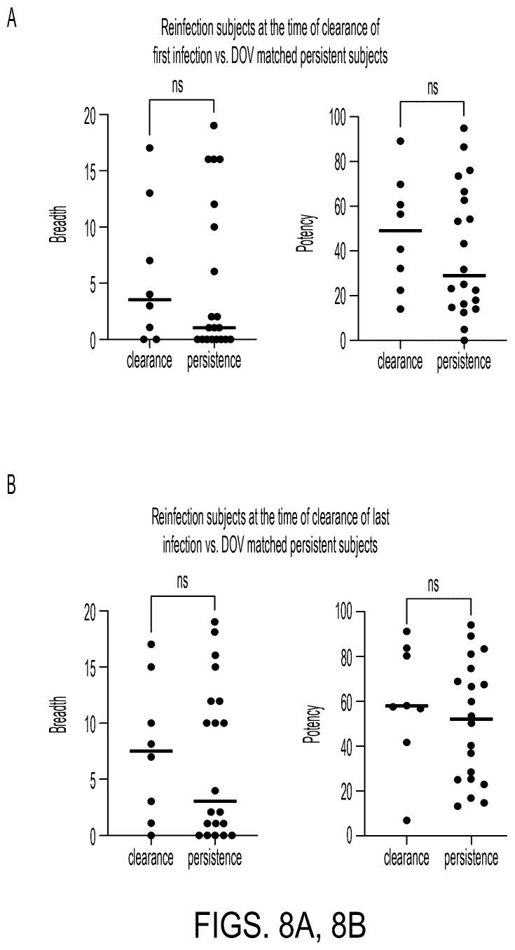

Plasma Neutralization of a Heterologous Virus Panel. The neutralizing activity of antibodies in participants' plasma obtained at longitudinal time points during infection was measured using a panel of 19 genotype 1a and 1b HCV pseudoparticles (HCVpp). This panel comprises HCVpp with a range of neutralization sensitivity expressing 94% of the amino acid polymorphisms present at greater than 5% frequency in a reference panel of 643 genotype 1 HCV isolates from GenBank [19]. The neutralizing breadth of mAbs measured with this genotype 1 panel and neutralizing breadth of the same mAbs measured with HCV strains from genotypes 1-6 was shown to be similar in previous studies [29, 40, 41]. Furthermore, a strong correlation was observed between breadth of plasma from HCV infected individuals measured with this panel of 19 genotype 1 HCVpp and breadth of the same plasma measured with an antigenically diverse panel discovered more recently which includes multiple genotypes (FIG. 7) [19, 37]. Plasma samples were collected at or immediately after the first viremic timepoint, prior to clearance of primary infection, prior to reinfection, immediately after reinfection and prior to clearance of reinfection (reinfection clearance group) or at days 587-1708 of reinfection (reinfection persistence group) (FIGS. 1A-1D). Samples were tested from persistence 1 strain and persistence strain switch subjects that were time-matched with reinfection subjects based on days of viremia (DOV) (FIGS. 2A-2B). Plasma antibody neutralizing breadth was defined as the number of HCVpp neutralized more than 25% by a 1:50 dilution of the plasma and potency as the highest percent neutralization value across the panel of 19 HCVpp by the same dilution of plasma. Close to 60% of subjects showed an increase in plasma neutralizing breadth and potency over the course of primary infection and reinfection, regardless of the outcome of infection, while the rest showed very low or no plasma antibody neutralization of the HCVpp panel (FIGS. 2A, 2B). There was a non-significant trend toward higher median breadth and potency for clearance samples compared to time-matched persistence samples (FIGS. 8A, 8B). Lack of statistical significance may be due to the small sample size of the groups. Persistence strain switch subjects had the highest median breadth and potency of all groups. This was true both when comparing overall breadth and potency of all time-matched plasma samples or highest breadth and potency for each subject (Table 1).

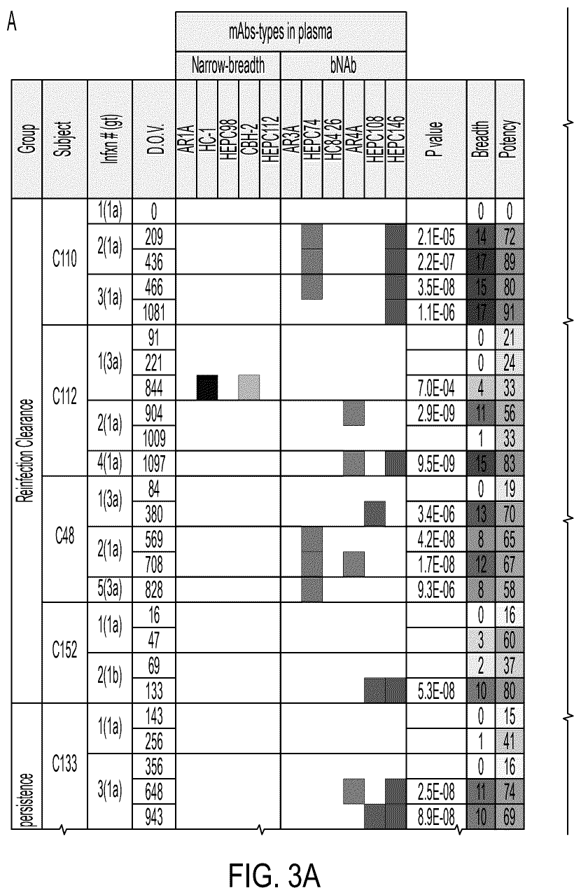

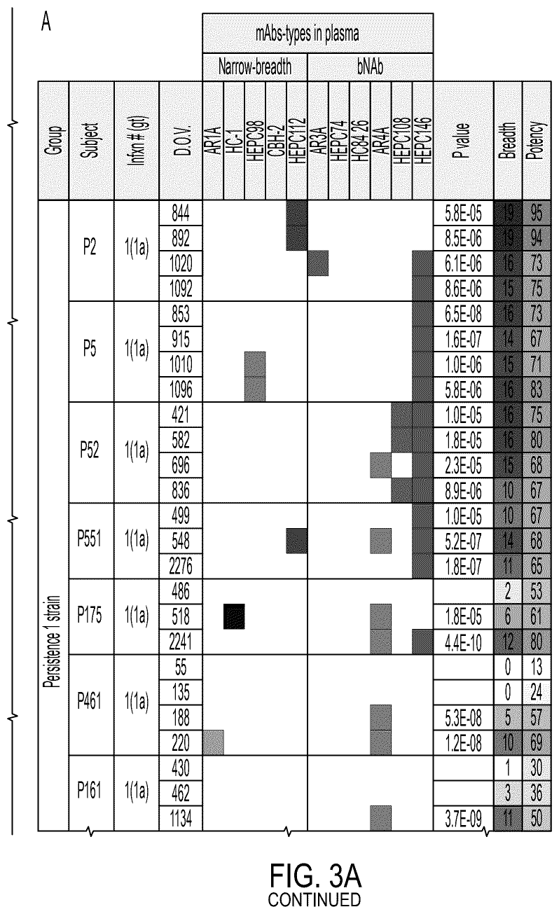

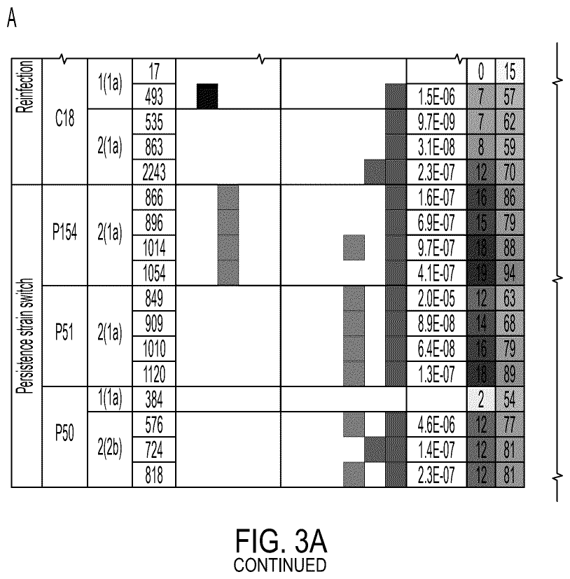

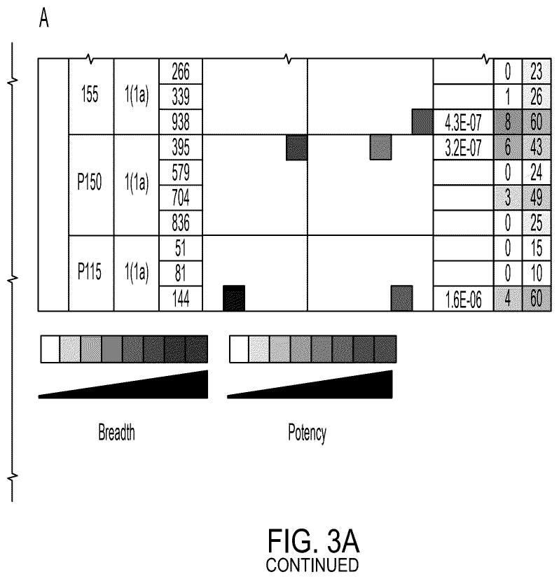

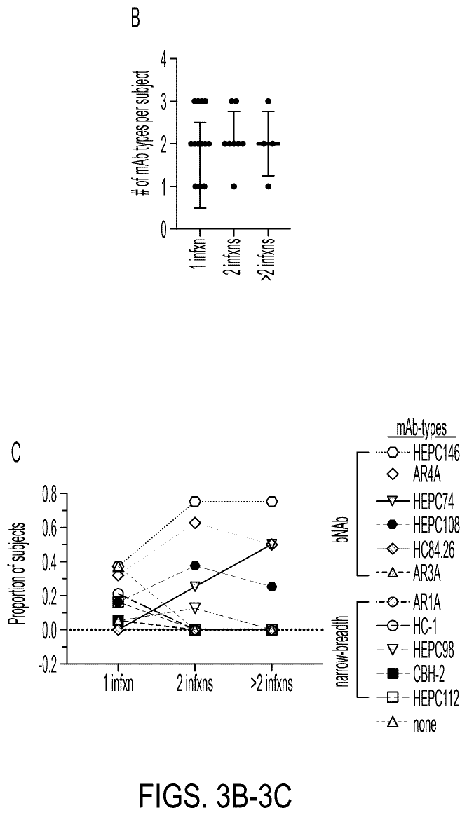

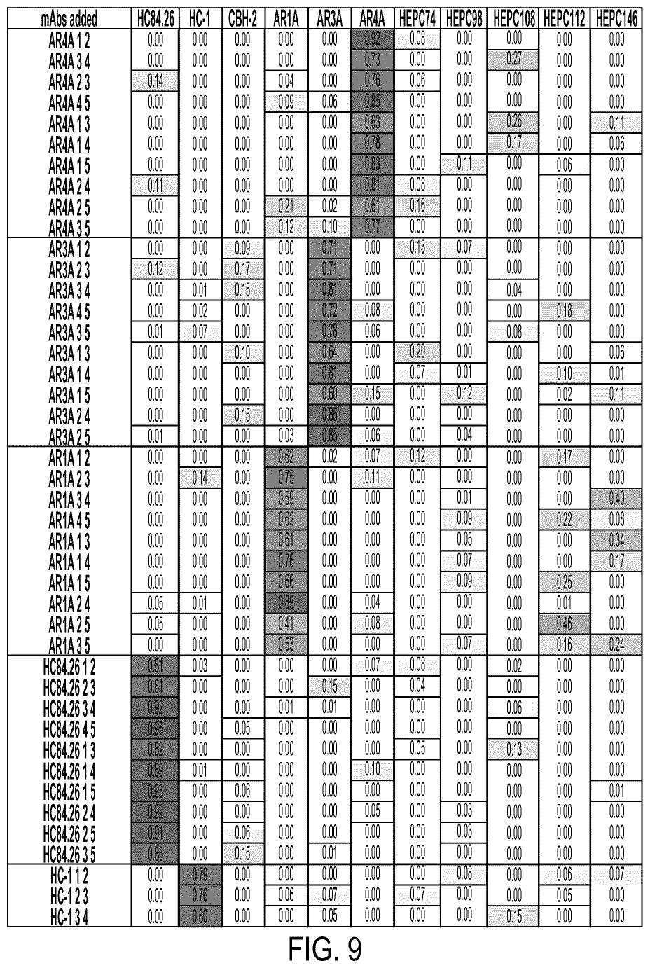

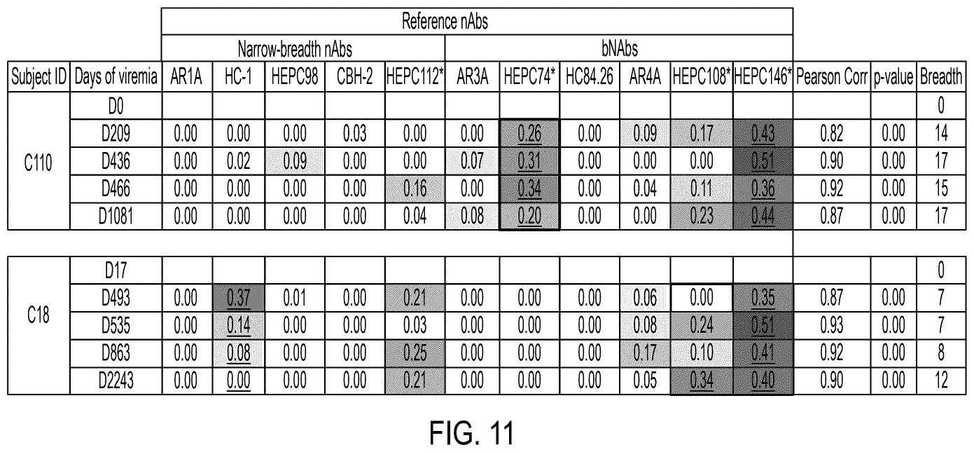

Deconvolution of NAb-Types in Plasma of HCV Infected Subjects. A plasma NAb deconvolution algorithm was applied to identify monoclonal antibody-types (mAb-types) responsible for the plasma neutralizing breadth and potency observed for each sample (FIG. 3A) [38]. A neutralization profile was generated for each plasma sample with breadth equal to or greater than four HCVpp by ranking its relative neutralization potency across the 19 HCVpp in the panel. By comparing these plasma neutralization profiles to the neutralization profiles of a panel of 11 E1E2-specific reference mAbs using code in Python, the combination of mAb-types present in each plasma sample was deconvoluted. The reference panel includes mAbs targeting neutralizing epitopes across E1 and E2 (AR1, AR3, AR4, HVR1, Domain B, Domain C, Domain D), and the previously described method was improved by expanding the panel of reference mAbs to include mAbs targeting three additional distinct antigenic sites: HEPC108 (a b. NAb that binds the E2 central beta sheet and front layer), HEPC146 (a bNAb that binds the E2 CD81 binding loop), and HEPC112 (a NAb that binds E1) [40]. Using deconvolution of control samples with spiked-in mAbs, the true positive threshold for identification of each reference mAb in monoclonal or polyclonal mixtures was determined. A mAb-type was called positive in test plasma samples only if its deconvolution value exceeded this threshold (FIG. 9). To validate the plasma deconvolution method after the addition of the new reference mAbs, a deconvolution analysis was performed on plasma of human subjects C110 and C18, from whom E1E2-specific mAbs from peripheral blood B cells had been previously isolated [29, 40]. As expected, HEPC74-type NAbs were identified in plasma of C110, the source of HEPC74-producing B cells. HEPC108-type and HEPC146-type NAbs were each identified in plasma of C18, the source of HEPC108 and HEPC146-producing B cells (FIG. 11).

Deconvolution demonstrated that plasma neutralizing breadth and potency observed in the study participants could be attributed to a variety of mAb-types in plasma (FIG. 3a). A median of two mAb-types per subject was detected during primary infection or subsequent reinfections (FIG. 3B). Notably, a larger variety of mAb-types was detected during primary infection, including some mAb-types with narrow neutralizing breadth. However, by the second or later infection of most subjects, only HEP146, AR4A, HEPC74 and HEPC108-like responses were detected. Taken together, these data show that some narrow-breadth mAb-types were present in plasma during primary infections, but these responses were superseded by four bNAb-types that became dominant during reinfections.

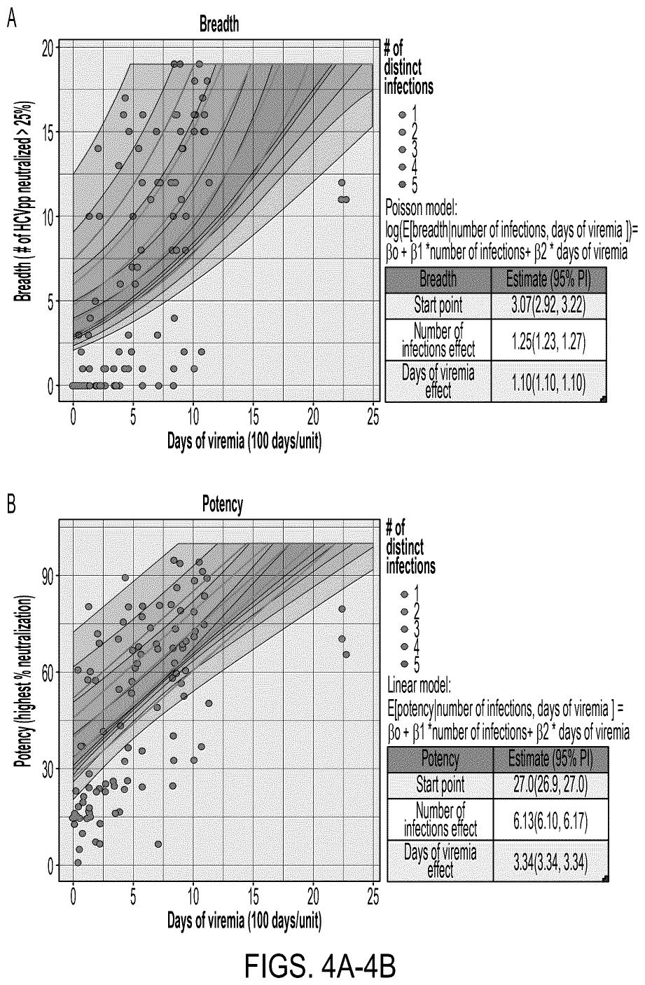

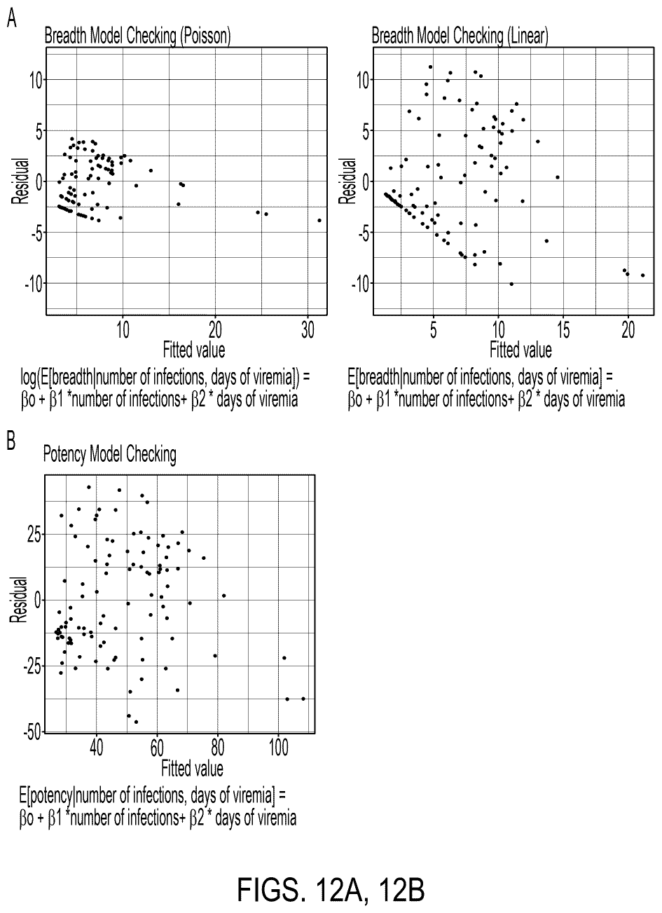

Duration of Viremia and Number of Distinct Infections are Associated with Greater Plasma Neutralizing Breadth and Potency Regardless of Infection Outcome. To identify variables associated with greater plasma neutralizing breadth, a regression analysis was performed to model the effect on neutralizing breadth and potency of duration of viremia, number of distinct infections (infections with divergence ≥0.03), and outcome of infection. The data set was divided into two subsets containing information from persistent or cleared infections. The days of viremia variable was scaled and measured in 100 days/unit. When conducting the regression analysis, the rate of neutralizing breadth increase was assumed to be constant with respect to days of viremia and was associated with the infection outcome and number of distinct infections per subject. These assumptions were tested using Poisson regression and linear regression. Since the residuals of the Poisson regression model showed smaller variance, the Poisson regression model was applied for evaluation of breadth (FIGS. 12A, 12B). The necessity of inclusion of outcome of infection was examined by conducting a F-test between the model with or without this variable. Since the two models were not significantly different (p=0.41), the outcome of infection variable was subsequently excluded from the prediction model for breadth. This indicates that clearance or persistence of infection was not significantly associated with breadth in the analysis. Instead, the Poisson regression model determined that neutralizing breadth was significantly associated with days viremia and number of distinct infections per subject (95% confidence interval of the effect coefficient does not cross 1 for either variable) (FIG. 4A).

Similar results were observed when modeling the effect of duration of viremia, number of distinct infections and outcome of infection on plasma neutralizing potency (FIG. 4B). In this case, the linear regression model was applied since the residuals were scattered and well spread. Here too, the models with or without the outcome of infection variable were not significantly different from one another (p=0.98). As with neutralizing breadth, the linear regression model demonstrated that neutralization potency was significantly associated with days viremia and number of distinct infections per subject (95% confidence interval of the effect coefficient does not cross 1 for either variable) (FIG. 4B). Therefore, duration of viremia and number of distinct infections are associated with greater plasma neutralizing breadth and potency in HCV infection regardless of whether the infection is cleared or persists.

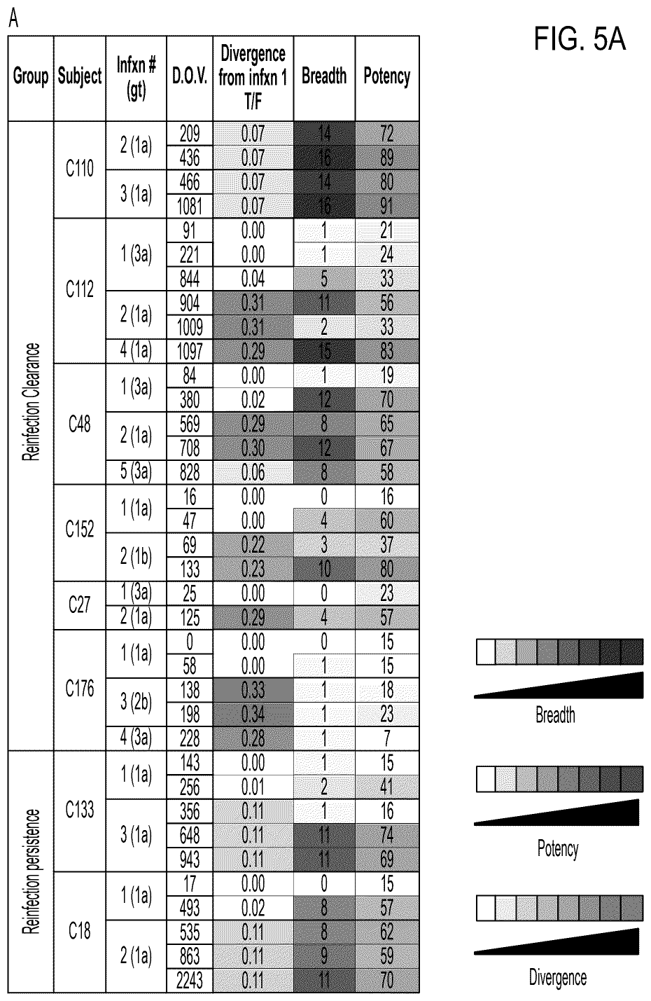

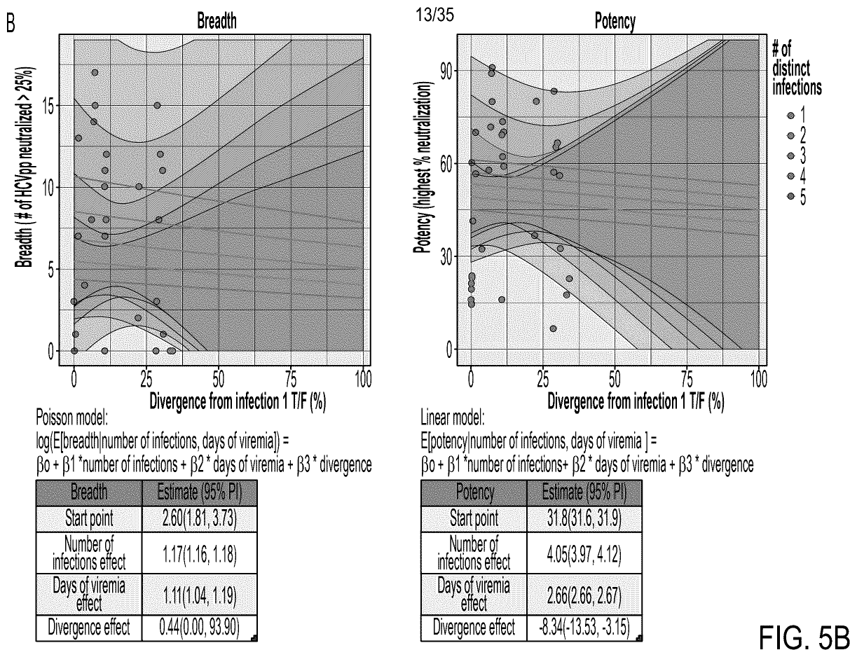

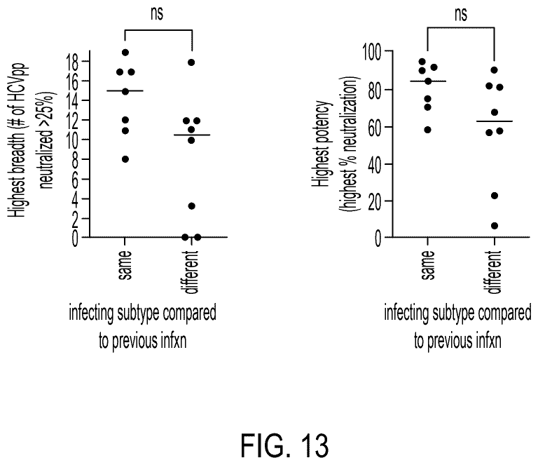

Greater Genetic Distance Between Infecting Viruses is not Associated with Greater Plasma Neutralizing Breadth or Potency. Next, it was evaluated whether greater genetic distance between primary infection and reinfection viruses was also associated with greater plasma neutralizing breadth or potency. First, plasma breadth and potency was compared during reinfections with the same or different HCV subtype as the primary infection. A nonsignificant trend toward higher breadth and potency was observed during reinfections with the same subtype as the previous infection (FIG. 13). Genetic distance was treated as a continuous variable by measuring divergence of reinfection E1E2 sequences from primary infection transmitted/founder (T/F) virus E1E2 of each subject (FIG. 5A). Divergence was determined by calculating the amino acid p distance between each reinfection E1E2 sequence and the most frequently observed primary infection T/F virus E1E2 sequence from the same subject. This analysis was limited to subjects from whom 5′ hemigenome sequences were previously obtained by single genome amplification (SGA) (n=8). The relationship was then modeled between E1E2 divergence of the re-infecting viruses, duration of viremia, number of distinct infections, and neutralizing breadth. The model was compared with and without genetic divergence via F-test and determined that inclusion of this variable did not significantly improve the model (p=0.85). The model including the genetic divergence variable was then used to make predictions about breadth. Greater genetic divergence of each re-infecting virus from the primary infection T/F virus was not associated with greater neutralizing breadth in reinfection subjects, as illustrated by the very large 95% confidence interval for the divergence effect coefficient (FIG. 5B). Similarly, when the effect of genetic divergence when predicting potency was evaluated, the F-test was not significant (p=0.83) and very large 95% confidence intervals for the divergence effect coefficient were observed (FIG. 5B). Therefore, greater genetic distance between primary and re-infecting viruses was not associated with greater neutralizing breadth or potency in reinfection subjects.

Repeated Infections with Antigenically Related, Antibody Sensitive Viruses were Associated with Greater Plasma Neutralizing Breadth and Potency. Since it was found that repeated exposure to highly genetically divergent infecting viruses was not associated with higher breadth or potency, the antigenicity of the infecting viruses was analyzed. To do so, binding was measured in an ELISA of a panel of E1E2-specific mAbs to longitudinal E1E2 proteins generated from viruses of eight of the study participants. This panel of mAbs included the mAbs used for neutralization profiling in FIGS. 3A-3C, along with the bNAb HC33.4 and the weakly neutralizing but broadly cross-reactive mAb CBH-7. In addition, it was hypothesized that E1E2 variants that were sensitive to binding of unmutated germline bNAb ancestors might play a role in early selection of bNAb-producing B cell lineages, so binding of inferred germline ancestors (recombinant unmutated ancestors (RUA)) of bNAbs HEPC146, HEPC74 and HEPC 108 was also measured [29].

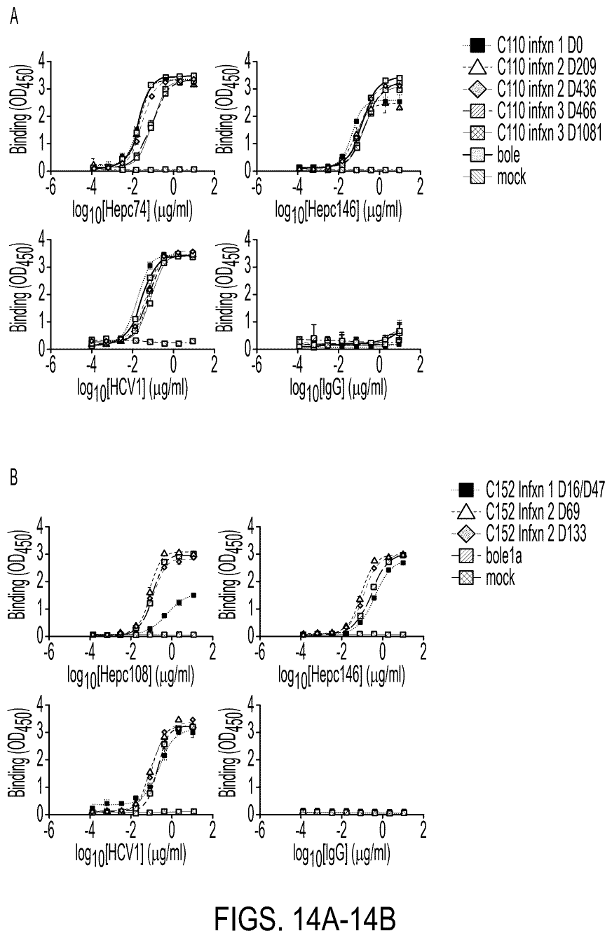

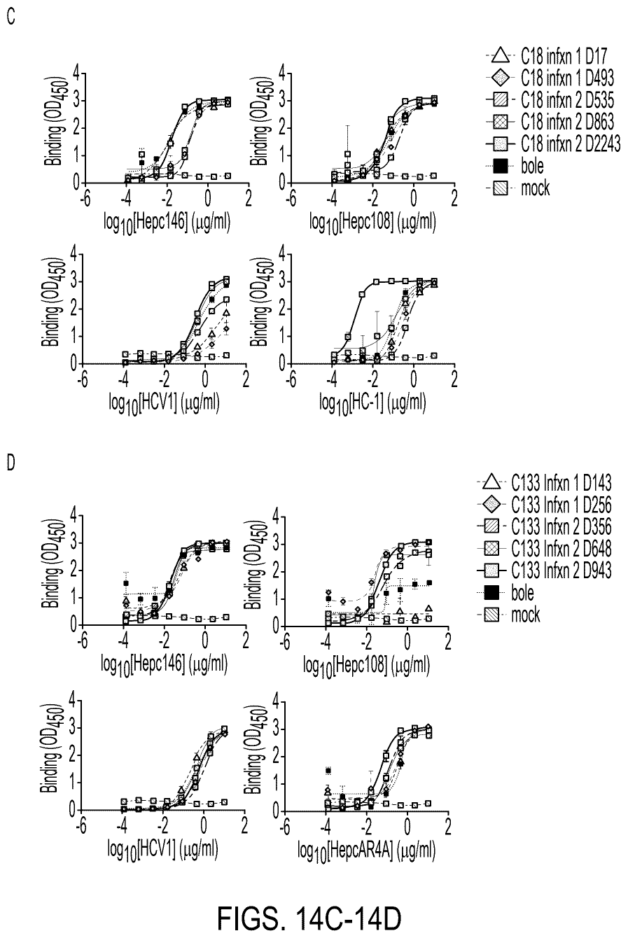

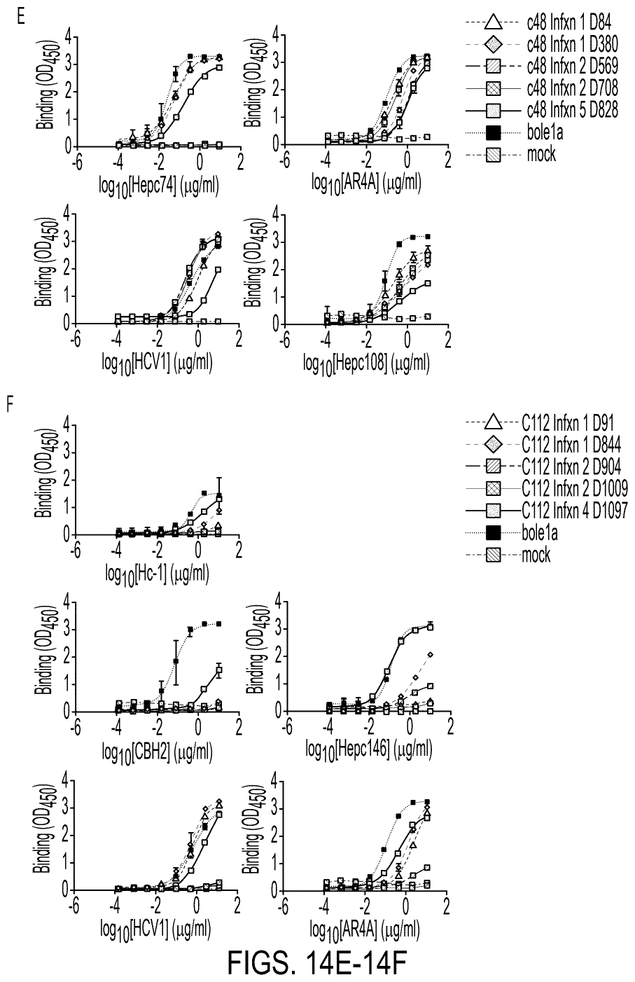

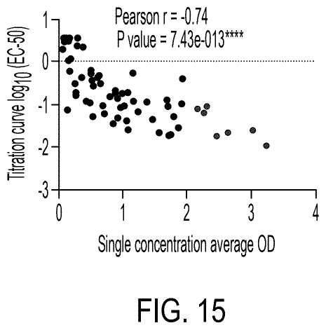

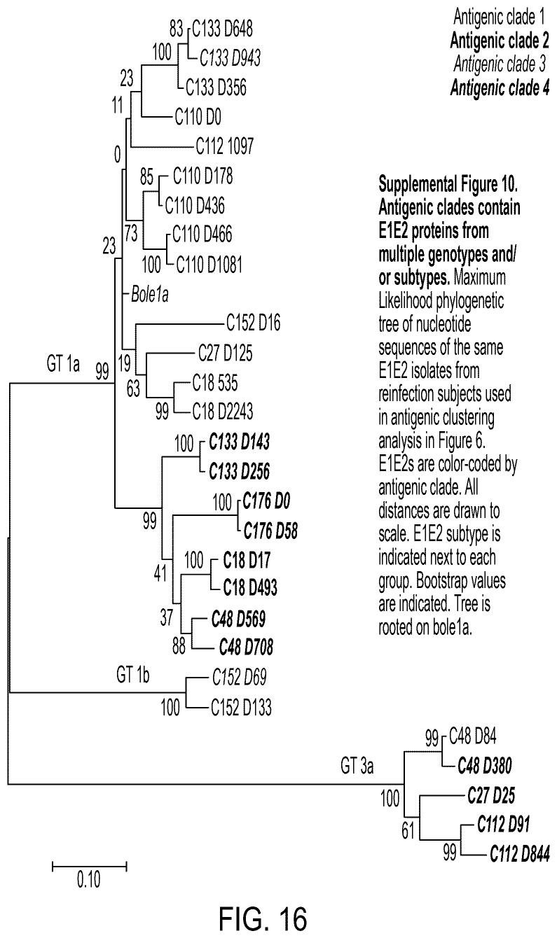

To select the optimal mAb concentration for binding quantitation, binding curves were first performed with serial dilutions of a subset of the reference mAbs and a subset of E1E2 proteins (FIGS. 14A-14F). From these curves, a single mAb concentration was identified (0.08 mg/mL) that fell in the exponential binding phase of the majority of binding curves. Subsequent mAb-E1E2 binding tests performed at this concentration. RUA mAbs were tested at a higher concentration because they were known from prior experiments to have low binding affinity (cite Flyak CHM). The decision to test most mAb-E1E2 combinations at a single mAb concentration was validated by the high correlation between the EC50 and optical density (OD) values for the mAb-E1E2 combinations that were tested both with full antibody titration curves and single mAb concentrations (p<0.0001, FIG. 15).

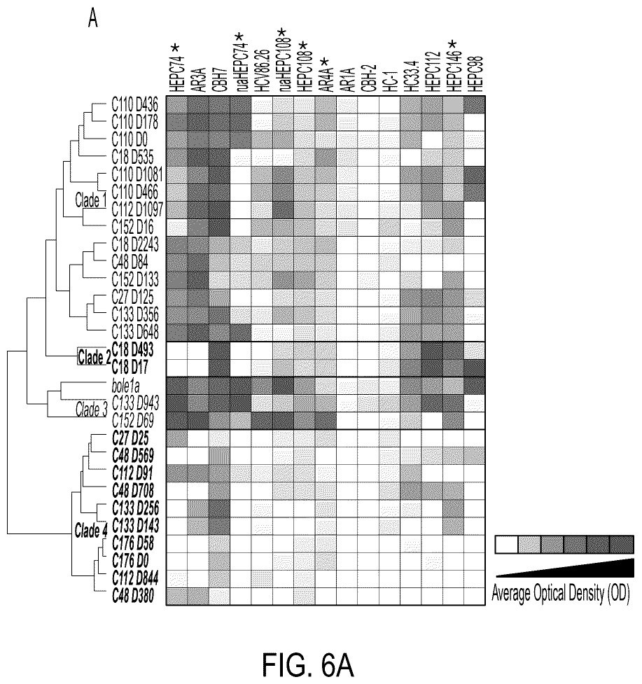

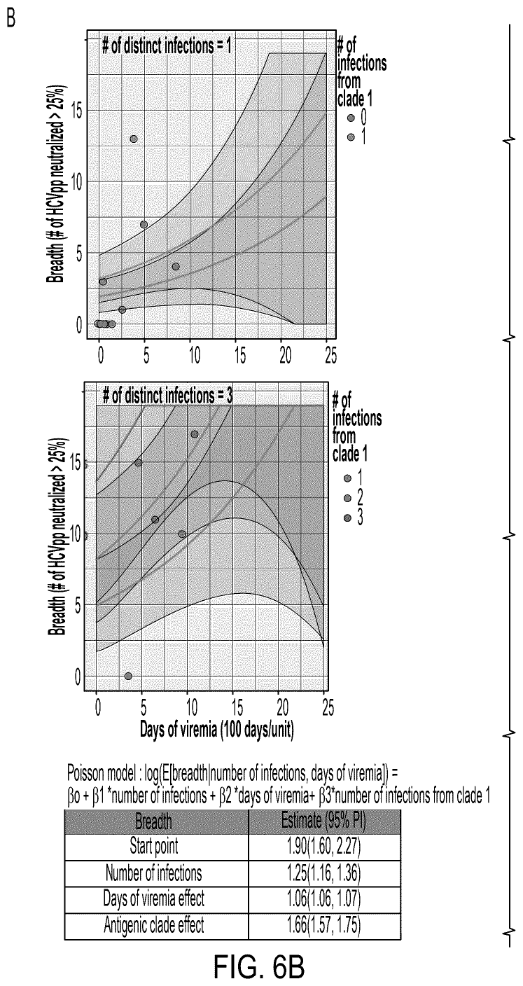

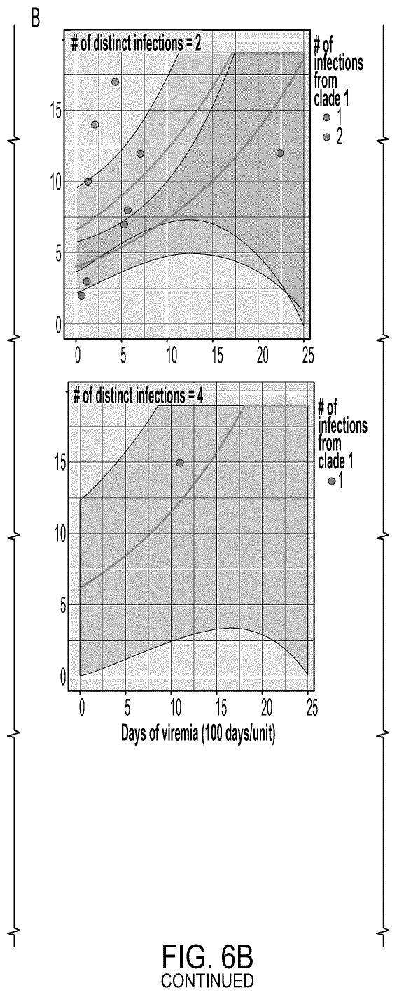

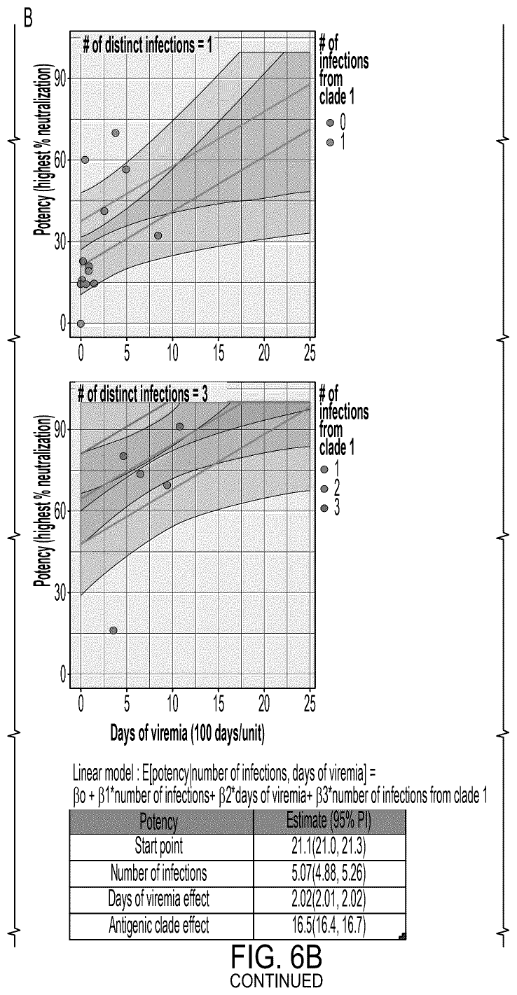

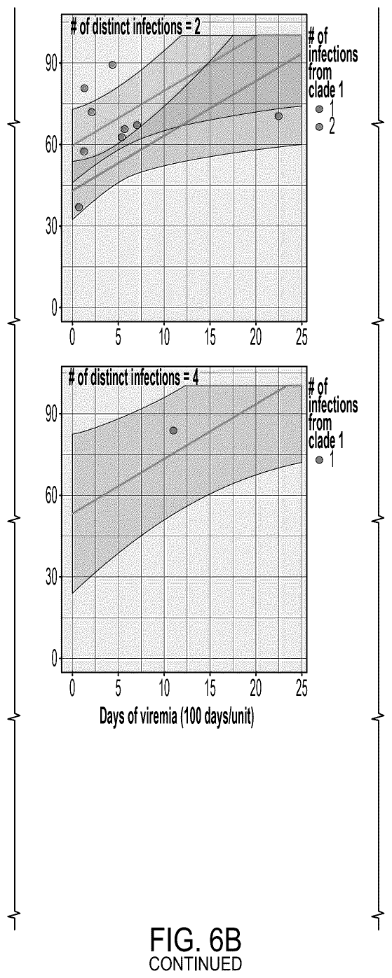

Binding of mAbs to longitudinal E1E2 proteins from reinfection subjects and to control E1E2 protein bole1a was measured. Bolela, a computationally designed ancestral genotype 1a HCV sequence, was included because it is a genetically representative strain that was demonstrated to be highly neutralization sensitive, with theoretical potential as a vaccine antigen [42-44]. The E1E2s clustered into 4 major antigenic clades (designated clades 1-4) based on their patterns of relative binding by all reference mAbs (FIG. 6A). Clade 1 included E1E2 proteins that were sensitive to the majority of reference mAbs in the panel. Clade 3 included bole1a and 2 other E1E2 proteins from reinfection subjects, which were also relatively sensitive to binding of the reference mAbs. E1E2 proteins in clades 2 and 4 were resistant to binding of the majority of mAbs. All clades, except clade 2, contained E1E2 proteins from multiple genotypes and/or subtypes (FIG. 16), indicating that the antigenic characteristics of the proteins were not dictated by their genotypes.

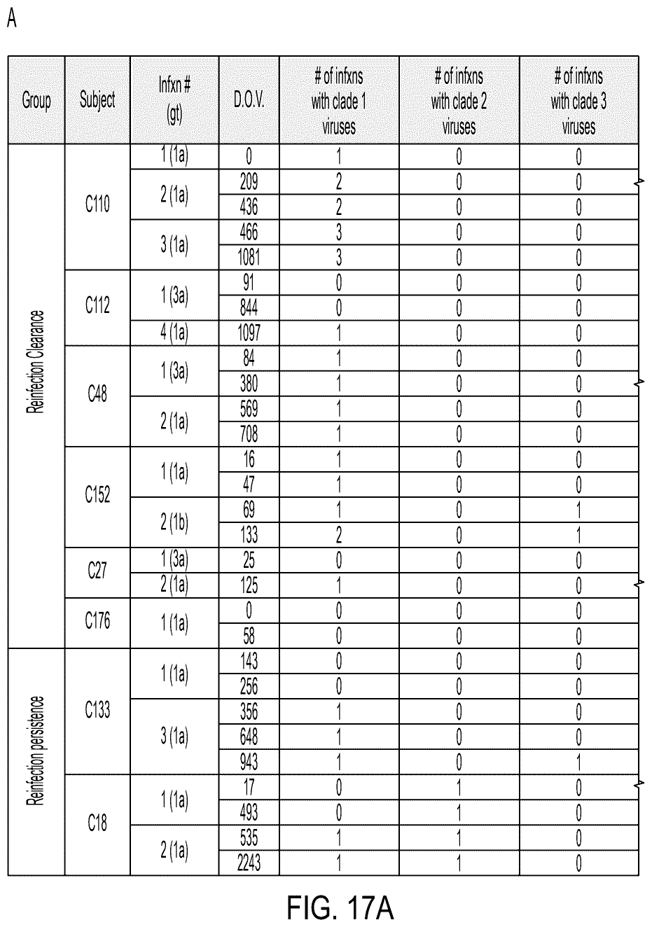

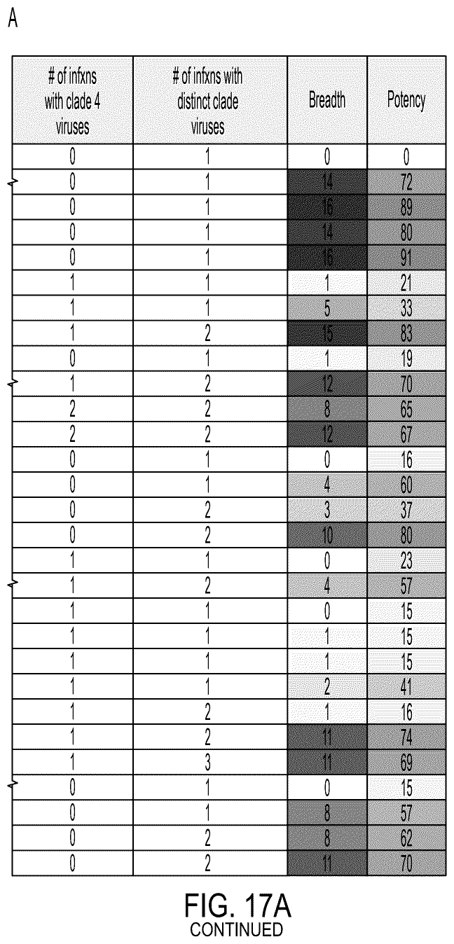

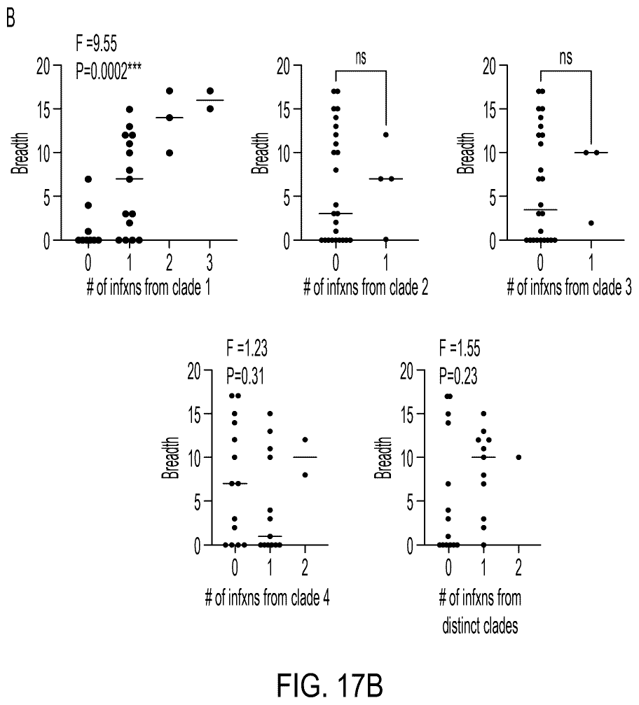

The relationship between infections with viruses from the different antigenic clades, neutralizing breadth, and potency was then explored. At each timepoint, the number of infections each subject had experienced with viruses from antigenic clades 1, 2, 3, or 4 as well as the number of infections with viruses from distinct antigenic clades, were counted and these values were compared to the neutralizing breadth of plasma from the same timepoints (FIG. 17A). It was found that only the number of distinct infections with viruses from antigenic clade 1 was significantly associated with greater neutralizing breadth (FIG. 17B). The relationship was then modeled between number of infections with distinct viruses from antigenic clade 1, duration of viremia, total number of infections, and neutralizing breadth (FIG. 6B). Although the total number of infections and the number of infections with viruses from antigenic clade 1 were highly correlated, inclusion of both variables was well tolerated by the model. The Poisson model showed that the number of infections with antigenic clade 1 variants was highly associated with greater neutralizing breadth, as illustrated by the high effect coefficient with a 95% confidence interval that did not cross 1. Similarly, a very high association between the number of infections with viruses expressing antigenic clade 1 E1E2 proteins and neutralizing potency was observed (FIG. 6B). Notably, antigenic clade 1 viruses were particularly sensitive to binding of the bNAbs that were immunodominant in broadly neutralizing plasma of reinfected individuals (HEPC146, AR4A, HEPC74, and HEPC108, FIGS. 3A-3C) as well as germline precursors of two of those bNAbs (HEPC74rua and HEPC108rua). In conclusion, it was found that repeated infections with antigenically related, antibody sensitive viruses, together with duration of viremia and total number of infections with genetically distinct viruses, were significantly associated with greater neutralizing breadth and potency.

TABLE 1
Participant Demographic and HCV Infection Data.

| | Reinfection | | Reinfection | | Persistence | | Persistence 1 | |
|---|---|---|---|---|---|---|---|---|
| | clearance | | persistence | | strain switch | | strain | |
| N of subjects | 6 | | 2 | | 3 | | 17 | |
| Age* | 24.7 | (2.9) | 29.5 | (9.2) | 23.3 | (4.7) | 25.25 | (2.7) |
| % Female | 33.3 | | 50 | | 33.3 | | 12.5 | |
| % Caucasian | 100 | | 100 | | 100 | | 100 | |
| HCV subtype (%) | | | | | | | | |
| 1a | 53.3 | | 100.0 | | 75.0 | | 88.2 | |
| 1b | 6.7 | | 0.0 | | 0 | | 0.0 | |
| 2b | 6.7 | | 0.0 | | 25.0 | | 5.8 | |
| 3a | 33.3 | | 0.0 | | 0 | | 5.8 | |
| Breadth** | 6 | (12.5) | 7 | (9.75) | 14.5 | (5.5) | 1.5 | (11) |
| Potency** | 57.7 | (52.3) | 59.7 | (34.6) | 79.9 | (17.1) | 36.7 | (48.1) |
| Highest breadth*** | 11.5 | (13.25) | 11.5 | (1) | 18 | (7) | 7 | (12.25) |
| Highest potency*** | 75.3 | (38.0) | 72.0 | (3.44) | 89.1 | (13.1) | 56.1 | (39.2) |

*At seroconversion, mean years (SD)
**Time-matched, median (IQR)
***For each subject across all infections, median (IQR)

### Discussion

Selection of HCV vaccine antigens that effectively elicit antibodies with strong neutralizing activity is critical. Here, key features of the antigenic stimuli capable of inducing potent anti-HCV bNAbs in humans were identified. The neutralizing breadth and potency of antibodies was measured in longitudinal plasma of each study participant and identified four major bNAb-types commonly induced upon reinfection. It was shown that the neutralizing breadth and potency of the antibody response increased upon repeated exposure to genetically distinct HCV strains and with longer duration viremia. It was also found that a specific antigenic profile of the infecting strains, not greater genetic difference between strains, was associated with increased breadth and potency in HCV reinfected subjects.

Induction of bNAbs is a major goal of HCV vaccine development. To date, the majority of candidate vaccines intended to induce bNAbs have relied on E1E2 antigens derived from a single virus or a combination of antigens selected to maximize genetic diversity. Unfortunately, these vaccines have failed to elicit high bNAb titers [45-48]. Therefore, there is a need for selection or design of more effective immunogens. E1E2 proteins from HCV-infected individuals were screened to identify possible antigenic differences between them and several were discovered that were sensitive to binding of reference mAbs targeting a diverse array of E2 epitopes. It was found that the number of infections with viruses harboring these pan-sensitive E1E2 proteins was highly associated with greater plasma neutralizing breadth and potency. Notably, E1E2 proteins in this pan-sensitive antigenic cluster were sensitive to the immunodominant bNAbs were identified in broadly neutralizing plasma of reinfected subjects, as well as germline precursors of two of those bNAbs. This observation would help define characteristics of antigens that should be included in an HCV vaccine.

A desirable vaccine will need to generate an immune response capable of neutralizing very diverse viruses across multiple genotypes. However, whether the viral genotype or genetic distance between infecting strains has significant influence on the development of such a broad response was previously unclear. Here, it was shown that greater genetic distance between infecting viruses was not associated with greater neutralizing breadth or potency, and that reinfection with a different HCV subtype from the primary infection did not broaden the neutralizing antibody response. Instead, the data point to repeated exposure to antigenically related, neutralization sensitive viruses as a better stimulus for bNAb induction. It is interesting to note that both antibody-sensitive and resistant antigenic clades included viruses from multiple genotypes, further indicating that genotypes do not dictate the antigenic characteristics of E1E2 proteins [37].

In agreement with prior studies, greater neutralizing breadth of the plasma antibody response was associated with longer duration of infection and with reinfection [19, 25-27]. Notably, in this study, antibody breath and potency was measured in plasma from multiple reinfections from the same subjects, including subjects who cleared as many as five distinct infections. Additionally, all plasma samples were time-matched based on days of viremia between the reinfection and persistence subjects. This allowed for consideration of the number of infections, duration of infection, and outcome of infection as separate variables in the model of bNAb induction. Lastly, the anti-HCV bNab-types induced in plasma were identified. By the second or later infection, a focusing of the immune response was observed towards HEP146, AR4A, HEPC74 and HEPC108-like responses (all broadly neutralizing) rather than narrow-breadth mAb-types. It is notable that AR4A and HEPC74-like responses in plasma have been previously associated with clearance of primary infection [38]. AR4A was recently shown to bind to the stalk of E2, while HEPC74 binds to the front layer/CD81 binding site of E2 [49, 50]. The two other immunodominant plasma b.NAbs, HEPC146 and HEPC108, were described more recently [40]. HEPC146 binding is focused at the CD81 binding loop of E2. The binding epitope of HEPC108 is less well defined but appears to include residues in both the central beta sheet and the front layer of E2. More work is needed to characterize these critical neutralizing epitopes.

Despite prior studies showing an association between early plasma neutralizing breadth and clearance of primary infection, a significant association was not observed in this study between plasma neutralizing breadth or potency and outcome of infection (clearance vs. persistence) [19, 20, 38]. Although induction of bNAbs is the goal for vaccine development, it is possible that clearance of infection is dictated to a greater extent by neutralization of autologous viruses, rather than neutralizing breadth against heterologous viruses that we measured in this study. Further work is needed to fully understand mechanisms of repeated control of HCV reinfections.

While a significant association of neutralizing breadth and potency was observed with repeated antigenic clade 1 exposure, it is worth noting that antigenic clade 3 E1E2 proteins were also sensitive to binding of most of the reference mAbs. The lack of association between number of antigenic clade 3 infections and breadth may have been dictated by the fact that this clade only included three E1E2 proteins, one of which was bole1a. Therefore, clade 3 E1E2 proteins might also be considered as possible vaccine antigens capable of eliciting a broad immune response.

In conclusion, key features of the stimuli associated with the induction of potent anti-HCV bNAbs was identified in humans. Data here indicate that longer duration of viremia and a greater number of infections are associated with greater plasma neutralizing breadth and potency. This broadening of the antibody response can be attributed to induction of four specific bNAb-types that we identified in the plasma upon reinfection. Repeated infection with antigenically related, antibody sensitive HCV strains was strongly associated with bNAb induction, while genetic distance between primary and reinfecting strains was less important. This study indicates that a prime-boost vaccine strategy with genetically distinct but antigenically similar, bNAb-sensitive E1E2 proteins, such as those in antigenic clades 1 and 3, should be considered as a vaccine strategy to induce potent bNAbs in humans.

### REFERENCES

1. WHO, Global Hepatitis Report, 2017. 2017, World Health Organization.
2. Terrault, N. A., Hepatitis C elimination: challenges with under-diagnosis and under-treatment. F1000Res, 2019. 8.
3. Grebely, J., et al., Elimination of HCV as a public health concern among people who inject drugs by 2030—What will it take to get there? J Int AIDS Soc, 2017. 20 (1): p. 22146.
4. Kanwal, F., et al., Risk of Hepatocellular Cancer in HCV Patients Treated With Direct-Acting Antiviral Agents. Gastroenterology, 2017. 153 (4): p. 996-1005.e1.
5. Hamdane, N., et al., HCV-Induced Epigenetic Changes Associated With Liver Cancer Risk Persist After Sustained Virologic Response. Gastroenterology, 2019. 156 (8): p. 2313-2329.e7.
6 Bailey, J. R., E. Barnes, and A. L. Cox, Approaches, Progress, and Challenges to Hepatitis C Vaccine Development. Gastroenterology, 2019. 156 (2): p. 418-430.
7. Bartenschlager, R., et al., Critical challenges and emerging opportunities in hepatitis C virus research in an era of potent antiviral therapy: Considerations for scientists and funding agencies. Virus Research, 2018. 248: p. 53-62.
8. Smith, D. B., et al., Expanded classification of hepatitis C virus into 7 genotypes and 67 subtypes: updated criteria and genotype assignment web resource. Hepatology, 2014. 59(1): p. 318-27.
9. Smith, D. B., et al., Proposed revision to the taxonomy of the genus Pestivirus, family Flaviviridae. J Gen Virol, 2017. 98 (8): p. 2106-2112.
10. Bukh, J., The history of hepatitis C virus (HCV): Basic research reveals unique features in phylogeny, evolution and the viral life cycle with new perspectives for epidemic control. J Hepatol, 2016. 65 (1 Suppl): p. S2-S21.
11. Law, M., et al., Broadly neutralizing antibodies protect against hepatitis C virus quasispecies challenge. Nat. Med., 2008. 14: p. 25-27.
12. Keck, Z. Y., et al., Affinity maturation of a broadly neutralizing human monoclonal antibody that prevents acute hepatitis C virus infection in mice. Hepatology, 2016. 64 (6): p. 1922-1933.
13. Giang, E., et al., Human broadly neutralizing antibodies to the envelope glycoprotein complex of hepatitis C virus. Proc Natl Acad Sci USA, 2012. 109 (16): p. 6205-10.
14. Morin, T. J., et al., Human Monoclonal Antibody HCV1 Effectively Prevents and Treats HCV Infection in Chimpanzees. PLOS. Pathog., 2012. 8: p. e1002895.
15. Hadlock, K. G., et al., Human monoclonal antibodies that inhibit binding of hepatitis C virus E2 protein to CD81 and recognize conserved conformational epitopes. J. Virol., 2000. 74: p. 10407-10416.
16. Kinchen, V. J., et al., Broadly Neutralizing Antibody Mediated Clearance of Human Hepatitis C Virus Infection. Cell Host Microbe, 2018. 24 (5): p. 717-730 e5.
17. Zibert, A., et al., Early antibody response against hypervariable region 1 is associated with acute self-limiting infections of hepatitis C virus. Hepatology, 1997. 25: p. 1245-1249.
18. Micallef, J. M., J. M. Kaldor, and G. J. Dore, Spontaneous viral clearance following acute hepatitis C infection: a systematic review of longitudinal studies. J Viral Hepat, 2006. 13 (1): p. 34-41.
19. Osburn, W. O., et al., Clearance of hepatitis C infection is associated with the early appearance of broad neutralizing antibody responses. Hepatology, 2014. 59 (6): p. 2140-51.
20. Pestka, J. M., et al., Rapid induction of virus-neutralizing antibodies and viral clearance in a single-source outbreak of hepatitis C. Proc. Natl. Acad. Sci. U.S.A, 2007. 104: p. 6025-6030.
21. Kinchen, V. J., A. L. Cox, and J. R. Bailey, Can Broadly Neutralizing Monoclonal Antibodies Lead to a Hepatitis C Virus Vaccine? Trends Microbiol, 2018. 26 (10): p. 854-864.
22. Hou, J., et al., Sequential immunization induces strong and broad immunity against all four dengue virus serotypes. npj Vaccines, 2020. 5 (1): p. 68.
23. Dong, W., et al., Cross-Protective Immune Responses Induced by Sequential Influenza Virus Infection and by Sequential Vaccination With Inactivated Influenza Vaccines. Frontiers in Immunology, 2018. 9.
24. Sprenger, K. G., et al., Optimizing immunization protocols to elicit broadly neutralizing antibodies. Proceedings of the National Academy of Sciences, 2020. 117 (33): p. 20077-20087.
25. Underwood, A. P., et al., Understanding the Determinants of BnAb Induction in Acute HCV Infection. Viruses, 2018. 10 (11): p. 659.
26. Logvinoff, C., et al., Neutralizing antibody response during acute and chronic hepatitis C virus infection. Proc. Natl. Acad. Sci. U.S.A, 2004. 101: p. 10149-10154.
27. Bartosch, B., et al., In vitro assay for neutralizing antibody to hepatitis C virus: evidence for broadly conserved neutralization epitopes. Proc. Natl. Acad. Sci. U.S.A, 2003. 100: p. 14199-14204.
28. Cox, A. L., et al., Prospective evaluation of community-acquired acute-phase hepatitis C virus infection. Clin. Infect. Dis., 2005. 40: p. 951-958.
29. Bailey, J. R., et al., Broadly neutralizing antibodies with few somatic mutations and hepatitis C virus clearance. JCI Insight, 2017. 2 (9).
30. Keck, Z. Y., et al., Human monoclonal antibodies to a novel cluster of conformational epitopes on HCV e2 with resistance to neutralization escape in a genotype 2a isolate. PLOS. Pathog., 2012. 8: p. e1002653.
31. Keck, Z., et al., Cooperativity in virus neutralization by human monoclonal antibodies to two adjacent regions located at the amino terminus of hepatitis C virus E2 glycoprotein. J. Virol., 2013. 87: p. 37-51.
32. Giang, E., et al., Human broadly neutralizing antibodies to the envelope glycoprotein complex of hepatitis C virus. Proc. Natl. Acad. Sci. U.S.A, 2012. 109: p. 6205-6210.
33. Rose, R., et al., Inconsistent temporal patterns of genetic variation of HCV among high-risk subjects may impact inference of transmission networks. Infection, Genetics and Evolution, 2019. 71: p. 1-6.
34. Hsu, M., et al., Hepatitis C virus glycoproteins mediate pH-dependent cell entry of pseudotyped retroviral particles. Proc. Natl. Acad. Sci. U.S.A, 2003. 100: p. 7271-7276.
35. Bailey, J. R., et al., Naturally selected hepatitis C virus polymorphisms confer broad neutralizing antibody resistance. J Clin Invest, 2015. 125 (1): p. 437-47.
36. Dowd, K. A., et al., Selection Pressure from Neutralizing Antibodies Drives Sequence Evolution during Acute Infection with Hepatitis C Virus. Gastroenterology, 2009. 136: p. 2377-2386.
37. Salas, J. H., et al., An Antigenically Diverse, Representative Panel of Envelope Glycoproteins for Hepatitis C Virus Vaccine Development. Gastroenterology, 2022. 162 (2): p. 562-574.
38. Kinchen, V. J., et al., Plasma deconvolution identifies broadly neutralizing antibodies associated with hepatitis C virus clearance. J Clin Invest, 2019. 130.
39. Pierce, B. G., et al., Global mapping of antibody recognition of the hepatitis C virus E2 glycoprotein: Implications for vaccine design. Proc Natl Acad Sci USA, 2016.
40. Colbert, M. D., et al., Broadly Neutralizing Antibodies Targeting New Sites of Vulnerability in Hepatitis C Virus E1E2. J Virol, 2019. 93(14).
41. Kinchen, V. J. and J. R. Bailey, Defining Breadth of Hepatitis C Virus Neutralization. Front Immunol, 2018. 9: p. 1703.
42. Munshaw, S., et al., Computational reconstruction of Bolela, a representative synthetic hepatitis C virus subtype 1a genome. J Virol, 2012. 86(10): p. 5915-21.
43. Burke, K. P., et al., Immunogenicity and cross-reactivity of a representative ancestral sequence in HCV infection. J. Immunol., 2012. 188: p. 5177-5188.
44. Wasilewski, L. N., et al., A Hepatitis C Virus Envelope Polymorphism Confers Resistance to Neutralization by Polyclonal Sera and Broadly Neutralizing Monoclonal Antibodies. J Virol, 2016. 90 (7): p. 3773-82.
45. Law, J. L., et al., A hepatitis C virus (HCV) vaccine comprising envelope glycoproteins gpE1/gpE2 derived from a single isolate elicits broad cross-genotype neutralizing antibodies in humans. PLOS One, 2013. 8(3): p. e59776.
46. Frey, S. E., et al., Safety and immunogenicity of HCV E1E2 vaccine adjuvanted with MF59 administered to healthy adults. Vaccine, 2010. 28: p. 6367-6373.
47. Houghton, M., Prospects for prophylactic and therapeutic vaccines against the hepatitis C viruses. Immunol Rev, 2011. 239(1): p. 99-108.
48. Liang, T. J., Current progress in development of hepatitis C virus vaccines. Nat Med, 2013. 19(7): p. 869-78.
49. Flyak, A. I., et al., HCV Broadly Neutralizing Antibodies Use a CDRH3 Disulfide Motif to Recognize an E2 Glycoprotein Site that Can Be Targeted for Vaccine Design. Cell Host Microbe, 2018. 24 (5): p. 703-716 e3.
50. de la Peña, A. T., et al., Structure of the hepatitis C virus E1E2 glycoprotein complex. bioRxiv, 2021: p. 2021.12.16.472992.

### Example 2

We have shown herein that HCV E1E2 variants could be grouped into antigenic clades based on their sensitivity to binding by a panel of mature and unmutated ancestor bNAbs. We found that repeated infection with bNAb-sensitive ‘antigenic clade 1’ viruses was associated with potent bNAb induction in humans, while infection with bNAb-resistant ‘antigenic clade 4’ viruses was associated with poor antibody induction. In this Example, we have identified polymorphisms that are responsible for the phenotypic differences between antigenic clade 1 and clade 4 E1E2.

Methods: We used the Subject-adjusted Neutralizing Antibody Prediction of Resistance (SNAPR) algorithm to identify significantly enriched polymorphisms in antigenic clades 1 or 4. Polymorphisms at six positions were predicted to mediate the antigenic difference between clades. We introduced clade 4 polymorphisms at each of these positions into a representative clade 1 E2 or E1E2 sequence using site-directed mutagenesis (SDM) and used ELISAs to measure the effect on binding of a panel of mature and unmutated ancestor bNAbs. Since H77 E1E2, a commonly used vaccine antigen, has clade 4 (potentially deleterious) polymorphisms at two of these positions, we also used SDM to convert these positions in H77 E1E2 to the clade 1 (potentially favorable) amino acid.

Results: Each substitution in the clade 1 E1E2 protein significantly decreased binding of at least one bNAb, and binding of each bNAb was significantly inhibited by at least one substitution. Three substitutions had a significant impact on binding across the entire bNAb panel-D431E, Q482D, and K500L. For H77 E1E2, binding of most mature front-layer-targeting bNAbs was increased by substitution E431D. Binding of most non-front layer bNAbs was increased by E482Q, and binding of all bNAbs was significantly improved by the substitutions in combination. Overall, the substitutions had a greater effect on the binding of unmutated ancestor bNAbs than the binding of mature bNAbs.

Conclusions: We identified polymorphisms that contribute to phenotypic differences between antigenic clades 1 and 4 E1E2s. Notably, the commonly used vaccine strain H77 has deleterious polymorphisms at two of these positions, and mutation of these positions to the favorable clade 1 polymorphism improved binding of many bNAbs, including bNAb unmutated ancestors. These data can allow optimization of vaccine antigens to favor binding of mature and germline ancestor bNAbs, which could enhance vaccine induction of these desirable antibodies.

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of identifying antigenic profiles of hepatitis C viruses comprising:
measuring plasma antibody neutralization of a heterologous hepatitis virus panel;
deconvoluting the plasma neutralizing antibodies to identify neutralizing antibodies having increased plasma neutralizing breadth and potency;
defining an antigenic profile of infecting hepatitis virus strains associated with increased breadth and potency in hepatitis C virus infected or reinfected subjects; thereby, identifying antigenic profiles of hepatitis C viruses.

2. The method of claim 1, wherein the neutralizing breadth and potency of the identified neutralizing antibodies is compared to control reference antibodies.

3. The method of claim 1, wherein the neutralizing breadth and potency of the identified neutralizing antibodies is determined by neutralization assays.

4. The method of claim 3, wherein the neutralization assays comprise generating pseudoviral particles and incubating the pseudoviral particles with target cells to determine infectivity of the pseudoviral particles.

5. The method of claim 4, wherein the pseudoviral particles identified as infectious are incubated with the biological sample and determining percent neutralization values of neutralizing antibodies from each biological sample.

6-7. (canceled)

8. The method of claim 1, wherein the neutralizing antibodies are quantitated and an antigenic profile is produced.

9. The method of claim 8, wherein the neutralizing antibodies are quantitated by an immunoassay.

10. The method of claim 1, wherein the hepatitis virus is hepatitis C virus (HCV).

11-12. (canceled)

13. A method of identifying immunogenic virus antigens comprising
extracting RNA from a biological sample comprising a viral infection;
quantifying and sequencing the RNA for defining specific viral strains;
conducting neutralization assays by incubating pseudoviral particles with target cells to determine infectivity and incubating the pseudoviral particles with a biological sample for determining percent neutralization for each biological sample;
converting of percent neutralization values to a neutralization profile for each biological sample;
averaging of plasma neutralization profiles to generate a final neutralization profile for each biological sample and comparing the neutralization profiles to reference profiles;
conducting a deconvolution analysis and selecting clones from various viremic time points;
quantitating antibody binding to viral antigens; thereby identifying immunogenic virus antigens.

14. The method of claim 13, wherein the virus is a hepatitis virus.

15. The method of claim 14, wherein the virus is a hepatitis C virus (HCV).

16. The method of claim 13, wherein the biological sample comprising an HCV infection is selected from earliest viremic and last viremic timepoints for each infection.

17. The method of claim 13, further comprising identifying neutralizing antibodies having increased plasma neutralizing breadth and potency as compared to control reference antibodies.

18-24. (canceled)

25. An isolated hybrid cell producing a Hepatitis C virus (HCV) neutralizing antibody identified by the method of claim 1.

26. A method of treating a subject infected with a Hepatitis C virus (HCV), comprising administering to the subject a therapeutically effective amount of one or more HCV neutralizing antibodies identified by the method of claim 1.

27-30. (canceled)

31. An immunogenic composition obtainable or obtained from a method of claim 24.

32. An immunogenic or vaccine composition comprising a polypeptide that comprises a hepatitis C virus (HCV) epitope which can induce an HCV neutralizing antibody.

33-35. (canceled)

36. The composition of claim 32, wherein the polypeptide comprises one or more amino acid substitutions at one or more positions 431, 482, 500 of HCV clade 1 E1E2 polypeptide.

37. The composition of claim 36, wherein the polypeptide comprises one or more amino acid substitutions at each of positions 431, 482, 500 of HCV clade 1 E1E2 polypeptide.

38. The composition of claim 36, wherein the one or more substitutions in the HCV clade 1 E1E2 protein comprise E43 ID or E482Q or the combination thereof.
